(19) United States
(12) Patent Application Publication
(10) Pub. No.: US 2012/0015029 A1
(43) Pub. Date: Jan. 19, 2012

(54) DIRECT COMPRESSION FORMULATION AND PROCESS

(76) Inventors: Sabine Pfeffer, Weil am Rhein (DE); Frank Schaefer, Reheinfelden (DE); Ricardo Schneeberger, Wentzwiller (FR); Paul Allen Sutton, Parsippany, NJ (US); Martin Friedrich Trueby, Rheinfelden (DE); Wolfgang Wirth, Arisdorf (DE)

(21) Appl. No.: 13/185,817

(22) Filed: Jul. 19, 2011

Related U.S. Application Data
(63) Continuation of application No. 11/814,274, filed on Jul. 18, 2007, now abandoned.
(60) Provisional application No. 60/644,645, filed on Jan. 18, 2005.
(60) Provisional application No. 60/690,484, filed on Jun. 14, 2005.

(51) Int. Cl. A61K 9/20; A61P 3/10; A61P 3/04; A61P 25/00; A61P 19/10; A61P 25/16; A61P 25/28; A61P 9/00; A61K 31/40; A61P 19/02

(52) U.S. Cl. 424/465; 424/464; 514/423; 428/402

(57) ABSTRACT

This invention relates to tablets especially tablets formed by direct compression of a dipeptidylpeptidase IV (DPP-IV) inhibitor compound, a process for the preparation thereof, to new pharmaceutical formulations, and new tableting powders comprising DPP-IV inhibitor formulations capable of being directly compressed into tablets. The invention relates further to a process for preparing the tablets by blending the active ingredient and specific excipients into the new formulations and then directly compressing the formulations into the direct compression tablets. The invention also relates to vildagliptin particle size distribution and a new crystal form of vildagliptin particularly adapted for the preparation of improved tablets and other pharmaceutical compositions.

This invention relates to tablets especially tablets formed by direct compression of a dipeptidylpeptidase IV (DPP-IV) inhibitor compound, a process for the preparation thereof, to new pharmaceutical formulations, and new tableting powders comprising DPP-IV inhibitor formulations capable of being directly compressed into tablets. The invention relates further to a process for preparing the tablets by blending the active ingredient and specific excipients into the new formulations and then directly compressing the formulations into the direct compression tablets. The invention also relates to vildagliptin particle size distribution and a new crystal form of vildagliptin particularly adapted for the preparation of improved tablets and other pharmaceutical compositions.

The preferred DPP-IV inhibitor compounds to which this invention is primarily directed are described below:

In the present context “a DPP-IV inhibitor” is also intended to comprise active metabolites and prodrugs thereof, such as active metabolites and prodrugs of DPP-IV inhibitors. A “metabolite” is an active derivative of a DPP-IV inhibitor produced when the DPP-IV inhibitor is metabolised. A “prodrug” is a compound that is either metabolised to a DPP-IV inhibitor or is metabolised to the same metabolite(s) as a DPP-IV inhibitor.

DPP-IV inhibitors are known in the art. For example, DPP-IV inhibitors are in each case generically and specifically disclosed e.g. in WO 98/19998, DE19616 486 A1, WO 00/34241, WO 95/15309, WO 01/72290, WO 01/52825, WO 9310127, WO 9925719, WO 9938501, WO 9946272, WO 9967278 and WO 9967279.

Preferred DPP-IV inhibitors are described in the following patent applications; WO 02053548 especially compounds 1001 to 1293 and examples 1 to 124, WO 02067918 especially compounds 1000 to 1278 and 2001 to 2159, WO 02066627 especially the described examples, WO 02/068420 especially all the compounds specifically listed in the examples I to LXIII and the described corresponding analogues, even preferred compounds are 2(28), 2(88), 2(119), 2(136) described in the table reporting IC50, WO 02083128 especially examples 1 to 13, US 2003096846 especially the specifically described compounds, WO 2004/037181 especially examples 1 to 33 and compounds of claims 3 to 5, WO 0168603 especially compounds of examples 1 to 109, EP1258480 especially compounds of examples 1 to 60, WO 0181337 especially examples 1 to 118, WO 02083109 especially examples 1A to 1D, WO 030003250 especially compounds of examples 1 to 166, most preferably 1 to 8, WO 03035067 especially the compounds described in the examples, WO 03/035057 especially the compounds described in the examples, US2003216450 especially examples 1 to 450, WO 99/46272 especially compounds of claims 12, 14, 15 and 17, WO 0197808 especially compounds of claim 2, WO 03002553 especially compounds of examples 1 to 33, WO 01/34594 especially the compounds described in the examples 1 to 4, WO 02051836 especially examples 1 to 712, EP1245568 especially examples 1 to 7, EP1258476 especially examples 1 to 32, US 2003087950 especially the described examples, WO 02/076450 especially examples 1 to 128, WO 03000180 especially examples 1 to 162, WO 03000181 especially examples 1 to 66, WO 03004498 especially examples 1 to 33, WO 0302942 especially examples 1 to 68, U.S. Pat. No. 6,482,844 especially the described examples, WO 0155105 especially the compounds listed in the examples 1 and 2, WO 0202560 especially examples 1 to 166, WO 03004496 especially examples 1 to 103, WO 03/024965 especially examples 1 to 54, WO 0303727 especially examples 1 to 209, WO 0368757 especially examples 1 to 88, WO 03074500 especially examples 1 to 72, examples 4.1 to 4.23, examples 5.1 to 5.10, examples 6.1 to 6.30, examples 7.1 to 7.23, examples 8.1 to 8.10, examples 9.1 to 9.30, WO 02038541 especially examples 1 to 53, WO 02062764 especially examples 1 to 293, preferably the compound of example 95 (2-{{3-(Aminomethyl)-4-butoxy-2-neopentyl-1-oxo-1,2 dihydro-6-isoquinolinyl}oxy}acetamide hydrochloride), WO 02308090 especially examples 1-1 to 1-109, examples 2-1 to 2-9, example 3, examples 4-1 to 4-19, examples 5-1 to 5-39, examples 6-1 to 6-4, examples 7-1 to 7-10, examples 8-1 to 8-8, examples 7-1 to 7-7 of page 90, examples 8-1 to 8-59 of pages 91 to 95, examples 9-1 to 9-33, examples 10-1 to 10-20, US 2003225102 especially compounds 1 to 115, compounds of examples 1 to 121, preferably compounds a) to z), aa) to az), ba) to bz), ca) to cz) and da) to dk), WO 0214271 especially examples 1 to 320 and US 2003096857, WO 2004/052850 especially the specifically described compounds such as examples 1 to 42 and compounds of claim 1, DE 102 56 264 A1 especially the described compounds such as examples 1 to 181 and the compounds of claim 5, WO 04/076433 especially the compounds specifically described, such as listed in table A, preferably the compounds listed in table B, preferably compounds I to XXXXVII, or compounds of claims 6 to 49, WO 04/071454 especially the specifically described compounds e.g. compounds 1 to 53 or compounds of tables Ia to If, or compounds of claims 2 to 55, WO 02/068420 especially the compounds specifically described, such as the compounds I to LXIII or Beispiele I and analogues 1 to 140 or Beispiele 2 and analogues 1 to 174 or Beispiele 3 and analogues 1, or Beispiele 4 to 5, or Beispiele 6 and analogues 1 to 5, or Beispiele 7 and analogues 1-3, or Beispiele 8 and analogue 1, or Beispiele 9, or Beispiele 10 and analogues 1 to 531 even preferred are compounds of claim 13, WO 03/000250 especially the compounds specifically described, such as the compounds 1 to 166, preferably compounds of examples 1 to 9, WO 03/024942 especially the compounds specifically described, such compounds 1 to 59, compounds of table 1 (1 to 68), compounds of claims 6, 7, 8, 9, WO 03024965 especially the compounds specifically described, such compounds 1 to 54, WO 03002593 especially the compounds specifically described, such compounds table 1 or of claims 2 to 15, WO 03037327 especially the compounds specifically described, such compounds of examples 1 to 209 WO 03/000250 especially the compounds specifically described, such as the compounds 1 to 166, preferably compounds of examples 1 to 9, WO 03/024942 especially the compounds specifically described, such compounds 1 to 59, compounds of table 1 (1 to 68), compounds of claims 6, 7, 8, 9, WO 03024965 especially the compounds specifically described, such compounds 1 to 54, WO 03002593 especially the compounds specifically described, such compounds table 1 or of claims 2 to 15, WO03037327 especially the compounds specifically described, such compounds of examples 1 to 209, WO 0238541, WO 0230890, U.S. application Ser. No. 09/788,173 filed Feb. 16, 2001 (attorney file LA50) especially the described examples, WO99/38501 especially the described examples, WO99/46272 especially the described examples and DE19616 486 A1 especially val-pyr, val-thiazolidide, isoleucyl-thiazolidide, isoleucyl-pyrrolidide, and fumar salts of isoleucyl-thiazolidide and isoleucyl-pyrrolidide, WO 0238541 especially the compounds specifically described, such compounds of examples 1 to 53, WO 03/002531 especially the compounds specifically described preferably the compounds listed on page 9 to 13, most preferably the compounds of examples 1 to 46 and even preferred compound of example 9, U.S. Pat. No. 6,395,767 preferably compound of examples 1 to 109 most preferably compound of example 60.

Further preferred DPP-IV inhibitors include the specific examples disclosed in U.S. Pat. No. 6,124,305 and U.S. Pat. No. 6,107,317, International Patent Applications, Publication Numbers WO 9819998, WO 95153 09 and WO 9818763; such as 1-[2-[(5-cyanopyridin-2-yl)aminoethylamino]acetyl-2-cyano-(S)-pyrrolidine and (2S)-I-[(2S)-2 amino-3,3-dimethylbutanoyl]-2-pyrrolidinecarbonitrile.

WO 9819998 discloses N—(N′-substituted glycyl)-2-cyano pyrrolidines, in particular 1-[2-[5-Cyanopyridin-2-yl]amino]-ethylamino]acetyl-2-cyano-(S)-pyrrolidine. Preferred compounds described in WO03/002553 are listed on pages 9 to 11 and are incorporated into the present application by reference. Published patent application WO 0034241 and published U.S. Pat. No. 6,110,949 disclose N-substituted adamantyl-amino-acetyl-2-cyano pyrrolidines and N-(substituted glycyl)-4-cyano pyrrolidines respectively. DPP-IV inhibitors of interest are specially those cited in claims 1 to 4. In particular these applications describe the compound 1-[[(3-Hydroxy-1-adamantyl)amino]acetyl]-2-cyano-(S)-pyrrolidine (also known as LAF237 or vildagliptin).

WO 9515309 discloses amino acid 2-cyanopyrrolidine amides as inhibitors of DPP-IV and WO 9529691 discloses peptidyl derivates of diesters of alpha-aminoalkylphosphonic acids, particularly those with proline or related structures. DPP-IV inhibitors of interest are specially those cited in Table 1 to 8. In WO 01/72290 DPP-IV inhibitors of interest are specially those cited in example 1 and claims 1, 4, and 6. WO 9310127 discloses proline boronic esters useful as DPP-IV inhibitors. DPP-IV inhibitors of interest are specially those cited in examples 1 to 19. Published patent application WO 9925719 discloses sulphostin, a DPP-IV inhibitor prepared by culturing a Streptomyces microorganism. WO 9938501 discloses N-substituted 4- to 8-membered heterocyclic rings. DPP-IV inhibitors of interest are specially those cited in claims 15 to 20.

WO 9946272 discloses phosphoric compounds as inhibitors of DPP-IV. DPP-IV inhibitors of interest are specially those cited in claims 1 to 23.

Other preferred DPP-IV inhibitors are the compounds of formula I, II or III disclosed in the patent application WO 03/057200 on page 14 to 27. Most preferred DPP-IV inhibitors are the compounds specifically described on pages 28 and 29.

Published patent applications WO 9967278 and WO 9967279 disclose DPP-IV prodrugs and inhibitors of the form A-B-C where C is either a stable or unstable inhibitor of DPP-IV.

Preferably, the N-peptidyl-O-aroyl hydroxylamine is a compound of formula VII

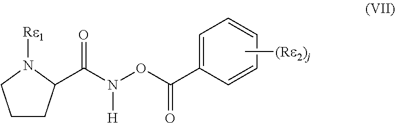

wherein
j is 0, 1 or 2;
Rε₁ represents the side chain of a natural amino acid; and
Rε₂ represents lower alkoxy, lower alkyl, halogen or nitro;
or a pharmaceutically acceptable salt thereof.

In a very preferred embodiment of the invention, the N-peptidyl-O-aroyl hydroxylamine is a compound of formula VIIa

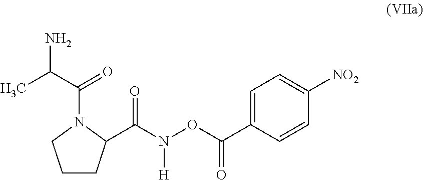

or a pharmaceutically acceptable salt thereof.

N-Peptidyl-O-aroyl hydroxylamines, e.g. of formula VII or VIIa, and their preparation are described by H. U. Demuth et al. in J. Enzyme Inhibition 1988, Vol. 2, pages 129-142, especially on pages 130-132.

Most preferably the inhibitors are N-(substituted glycyl)-2-cyanopyrrolidines of formula (I)

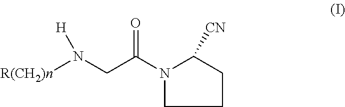

wherein

R is substituted adamantyl; and

n is 0 to 3; in free form or in acid addition salt form.

The term “substituted adamantly” refers to adamantyl, i.e., 1- or 2-adamantyl, substituted by one or more, e.g., two substituents selected from alkyl, —OR₁ or —NR₂R₃, where R₁, R₂ and R₃ are independently hydrogen, alkyl, (C₁-C₈alkanoyl), carbamyl, or —CO—NR₄R₅, where R₄ and R₅ are independently alkyl, unsubstituted or substituted aryl and where one of R₄ and R₅ additionally is hydrogen or R₄ and R₅ together represent C₂-C₇alkylene.

The term “aryl” preferably represents phenyl. Substituted phenyl preferably is phenyl substituted by one or more, e.g., two, substitutents selected from, e.g., alkyl, alkoxy, halogen and trifluoromethyl.

The term “alkoxy” refers to alkyl-O—.

The term “halogen” or “halo” refers to fluorine, chlorine, bromine and iodine.

The term “alkylene” refers to a straight chain bridge of 2 to 7 carbon atoms, preferably of 3 to 6 carbon atoms, most preferably 5 carbon atoms.

A preferred group of compounds of the invention is the compounds of formula (I), wherein the substituent on the adamantyl is bonded on a bridgehead or a methylene adjacent to a bridgehead. Compounds of formula (I), wherein the glycyl-2-cyanopyrrolidine moiety is bonded to a bridgehead, the R′ substituent on the adamantyl is preferably 3-hydroxy. Compounds of formula (I), wherein the glycyl-2-cyanopyrrolidine moiety is bonded at a methylene adjacent to a bridgehead, the R′ substituent on the adamantyl is preferably 5-hydroxy.

The present invention especially relates to a compound of formula (IA) or (IB)

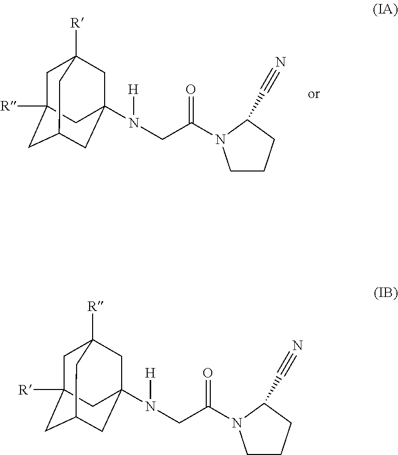

wherein
R′ represents hydroxy, C₁-C₇alkoxy, C₁-C₈alkanoyloxy or R₅R₄N—CO—O—, where R₄ and R₅ independently are C₁-C₇alkyl or phenyl which is unsubstituted or substituted by a substitutent selected from C₁-C₇alkyl, C₁-C₇alkoxy, halogen and trifluoromethyl and where R₄ additionally is hydrogen; or R₄ and R₅ together represent C₃-C₆alkylene; and
R″ represents hydrogen; or
R′ and R″ independently represent C₁-C₇alkyl;
   in free form or in form of a pharmaceutically acceptable acid addition salt.

These DPP-IV inhibitor compounds of formula (I), (IA) or (IB) are known and described in U.S. Pat. No. 6,166,063, issued Dec. 26, 2000 and WO 01/52825. Specially disclosed is (S)-1-{2-[5-cyanopyridin-2-yl)amino]ethyl-aminoacetyl)-2-cyano-pyrrolidine or (S)-1-[(3-hydroxy-1 adamantyl)amino]acetyl-2-cyano-pyrrolidine (LAF237 or vildagliptin). They can exist in free form or in acid addition salt form. Pharmaceutically acceptable, i.e., non-toxic and physiologically acceptable, salts are preferred, although other salts are also useful, e.g., in isolating or purifying the compounds of this invention. Although the preferred acid addition salts are the hydrochlorides, salts of methanesulfonic, sulfuric, phosphoric, citric, lactic and acetic acid may also be utilized.

Preferred DPP-IV inhibitors are those described by Mona Patel and col. (Expert Opinion Investig Drugs. 2003 April; 12(4):623-33) on the paragraph 5, especially P32/98, K-364, FE-999011, BDPX, NVP-DDP-728 and others, which publication is hereby incorporated by reference especially the described DPP-IV inhibitors.

FE-999011 is described in the patent application WO 95/15309 page 14, as compound No. 18.

Another preferred inhibitor is the compound BMS-477118 disclosed in U.S. Pat. No. 6,395,767 (compound of example 60) also known as is (1S,3S,5S)-2-[(2S)-2-amino-2-(3-hydroxytricyclo[3.3.1.1^{3,7}]dec-1-yl)-1-oxoethyl]-2-azabicyclo[3.1.0]hexane-3-carbonitrile, benzoate (1:1) as depicted in Formula M of the patent application WO 2004/052850 on page 2, and the corresponding free base, (1S,3S,5S)-2-[(2S)-2-amino-2-(3-hydroxy-tricyclo[3.3.1.1^{3,7}]dec-1-yl)-1-oxoethyl]-2-azabicyclo-[3.1.0]hexane-3-carbonitrile (M′) and its monohydrate (M″) as depicted in Formula M of the patent application WO 2004/052850 on page 3.

Another preferred inhibitor is the compound GSK23A disclosed in WO 03/002531 (example 9) also known as (2S,4S)-1-((2R)-2-Amino-3-[(4-methoxybenzyl)sulfonyl]-3-methylbutanoyl)-4-fluoropyrrolidine-2-carbonitrile hydrochloride.

Other very preferred DPP-IV inhibitors of the invention are described in the International patent application WO 02/076450 (especially the examples 1 to 128) and by Wallace T. Ashton (Bioorganic & Medicinal Chemistry Letters 14 (2004) 859-863) especially the compound 1 and the compounds listed in the tables 1 and 2. The preferred compound is the compound 21e (table 1) of formula

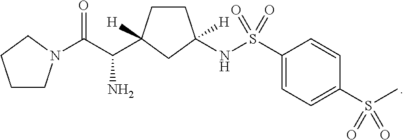

P32/98 or P3298 (CAS number: 251572-86-8) also known as 3-[(2S,35)-2-amino-3-methyl-1-oxopentyl]thiazolidine can be used as 3-[(2S,3S)-2-amino-3-methyl-1-oxopentyl]thiazolidine and (2E)-2-butenedioate (2:1) mixture such as shown below

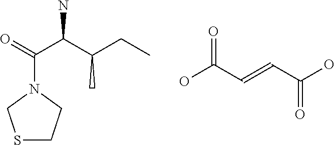

and is described in WO 99/61431 in the name of Probiodrug and also the compound P 93/01.

Other preferred DPP-IV inhibitors are the compounds disclosed in the patent application WO 02/083128 such as in the claims 1 to 5. Most preferred DPP-IV inhibitors are the compounds specifically described by the examples 1 to 13 and the claims 6 to 10.

Other preferred DPP-IV inhibitors are described in the patent applications WO 2004/037169 especially those described in the examples 1 to 48 and WO 02/062764 especially the described examples 1 to 293, even preferred are the compounds 3-(aminomethyl)-2-isobuthyl-1-oxo-4-phenyl-1,2-dihydro-6-isoquinolinecarboxamide and 2-{[3-(aminomethyl)-2-isobuthyl-4-phenyl-1-oxo-1,2-dihydro-6-isoquinolyl]oxy}acetamide described on page 7 and also in the patent application WO2004/024184 especially in the reference examples 1 to 4.

Other preferred DPP-IV inhibitors are described in the patent application WO 03/004498 especially examples 1 to 33 and most preferably the compound of the formula

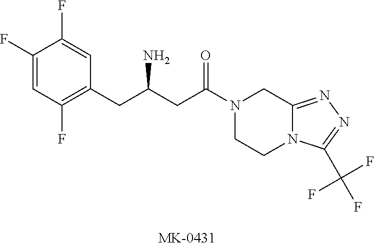

described by the example 7 and also known as MK-0431.

Preferred DPP-IV inhibitors are also described in the patent application WO 2004/037181 especially examples 1 to 33, most preferably the compounds described in the claims 3 to 5.

Preferred DPP-IV inhibitors are N-substituted adamantyl-amino-acetyl-2-cyano pyrrolidines, N (substituted glycyl)-4-cyano pyrrolidines, N—(N′-substituted glycyl)-2-cyanopyrrolidines, N-aminoacyl thiazolidines, N-aminoacyl pyrrolidines, L-allo-isoleucyl thiazolidine, L-threo-isoleucyl pyrrolidine, and L-allo-isoleucyl pyrrolidine, 1-[2-[(5-cyanopyridin-2-yl)amino]ethylamino]acetyl-2-cyano-(S)-pyrrolidine and pharmaceutical salts thereof.

Especially preferred are 1-{2-[(5-cyanopyridin-2-yl)amino]ethylamino}acetyl-2 (S)-cyano-pyrrolidine dihydrochloride (DPP728), of formula

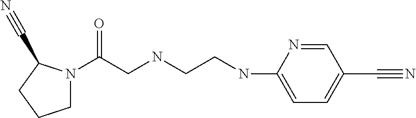

especially the dihydrochloride thereof,
and (S)-1-[(3-hydroxy-1-adamantyl)amino]acetyl-2-cyano-pyrrolidine (LAF237 or vildagliptin (Non-proprietary name—INN)) of formula

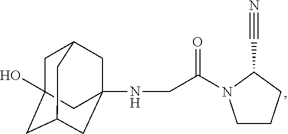

and L-threo-isoleucyl thiazolidine (compound code according to Probiodrug: P32/98 as described above), MK-0431, GSK23A, BMS-477118, 3-(aminomethyl)-2-isobuthyl-1-oxo-4-phenyl-1,2-dihydro-6-isoquinolinecarboxamide and 2-{[3-(aminomethyl)-2-isobuthyl-4-phenyl-1-oxo-1,2-dihydro-6-isoquinolyl]oxy}acetamide and optionally in any case pharmaceutical salts thereof.

DPP728 and LAF237 are the very preferred compounds and are specifically disclosed in Example 3 of WO 98/19998 and Example 1 of WO 00/34241, respectively. The DPP-IV inhibitor P32/98 (see above) is specifically described in Diabetes 1998, 47, 1253-1258. DPP728 and LAF237 can be formulated as described on page 20 of WO 98/19998 or in WO 00/34241. The preferred formulations for the administration of LAF237 are described in the U.S. provisional application No. 60/604,274. In the present application, the term “vildagliptin” refers to any form of vildagliptin such as amorphous vildagliptin, crystalline forms of vildagliptin, crystalline form “A” of vildagliptin, a partially crystalline form of vildagliptin, a polymorphous form of vildagliptin, a solvate form of vildagliptin or an hydrate form of vildagliptin, or any salt thereof.

In the herein application, when the applicant refers to 100 mg of vildagliptin he refers to 100 mg of the free base, or a respective amount of a pharmaceutically acceptable salt thereof.

### BRIEF DESCRIPTION OF THE DRAWINGS

FIG. 1 summarizes an X-Ray powder diffraction pattern for vildagliptin (form A).

FIG. 2 summarizes an Infrared spectrum for vildagliptin (form A).

FIG. 3 summarizes a pharmokinetic profile for different dosages of vildagliptin in patients.

FIG. 4 summarizes Cmax and AUC data for different dosages of vildagliptin in patients.

FIG. 5 summarizes blood plasma concentrations of vildagliptin (alone and in combination with metformin) in Type 2 diabetic patients.

FIG. 6 summarizes blood plasma concentrations of vildagliptin (alone and in combination with pioglitazone) in Type 2 diabetic patients.

FIG. 7 summarizes DPP-4 inhibition for different dosages of vildagliptin in patients after a single oral administration of each dosage.

Especially preferred are orally active DPP-IV inhibitors.

In each case in particular in the compound claims and the final products of the working examples, the subject matter of the final products, the pharmaceutical preparations and the claims are hereby incorporated into the present application by reference to the herein mentioned publications or patent applications.

The DPP-IV inhibitor compounds e.g. those of formula (I), and their corresponding pharmaceutically acceptable acid addition salts, may be combined with one or more pharmaceutically acceptable carriers and, optionally, one or more other conventional pharmaceutical adjuvants and administered enterally, e.g., orally, in the form of tablets, capsules, caplets, etc. or parenterally, e.g., intravenously, in the form of sterile injectable solutions or suspensions. The enteral and parenteral compositions may be prepared by conventional means.

The DPP-IV inhibitor compounds e.g. those of formula (I), and their corresponding pharmaceutically acceptable acid addition salts, may be formulated into enteral and parenteral pharmaceutical compositions containing an amount of the active substance that is effective for treating conditions mediated by DPP-IV inhibition, such compositions in unit dosage form and such compositions comprising a pharmaceutically acceptable carrier.

The DPP-IV inhibitor compounds e.g. those of formula (I), including those of each of the sub-scopes thereof and each of the examples, may be administered in enantiomerically pure form, e.g., >98%, preferably >99%; or together with the R enantiomer, e.g., in racemic form. The above dosage ranges are based on the compounds of formula (I), excluding the amount of the R enantiomer.

In view of their ability to inhibit DPP-IV, the DPP-IV inhibitor compounds e.g. those of formula (I), and their corresponding pharmaceutically acceptable acid addition salts, are useful in treating conditions mediated by DPP-IV inhibition. Based on the above and findings in the literature, it is expected that the compounds disclosed herein are useful in the treatment of conditions, such as non-insulin-dependent diabetes mellitus, arthritis, obesity, allograft transplantation and calcitonin-osteoporosis. In addition, based on the roles of glucagon-like peptides, such as GLP-1 and GLP-2, and their association with DPP-IV inhibition, it is expected that the compounds disclosed herein are useful for example, to produce a sedative or anxiolytic effect, or to attenuate post-surgical catabolic changes and hormonal responses to stress, or to reduce mortality and morbidity after myocardial infarction, or in the treatment of conditions related to the above effects which may be mediated by GLP-1 and/or GLP-2 levels.

More specifically, e.g., the DPP-IV inhibitor compounds e.g. those of formula (I), and their corresponding pharmaceutically acceptable acid addition salts, improve early insulin response to an oral glucose challenge and, therefore, are useful in treating non-insulin-dependent diabetes mellitus.

The DPP-IV inhibitor compounds especially compounds of formula I, IA or IB, useful in this invention are hygroscopic, presents stability problems, and are not inherently compressible. Consequently, there is a need to provide a free-flowing and cohesive composition capable of being directly compressed into strong tablets with an acceptable in vitro dissolution profile. Tablets may be defined as solid dosage pharmaceutical forms containing drug substances with or without suitable fillers. They are produced by compression or compaction of a formulation containing the active ingredient and certain excipients selected to aid in the processing and to improve the properties of the product. Tablets may be coated or uncoated and are made from powdered, crystalline materials. They may include various diluents, binders, disintegrants, lubricants, glidants and in many cases, colorants. Excipients used are classified according to the function they perform. For example, a glidant may be used to improve the flow of powder blend in the hopper and into the tablet die.

There has been widespread use of tablets and the majority of pharmaceutical dosage forms are marketed as tablets. Major reasons of tablet popularity as a dosage form are simplicity, low cost and the speed of production. Other reasons include stability of drug product, convenience in packaging, shipping and dispensing. To the patient or consumer, tablets offer convenience of administration, ease of accurate dosage, compactness, portability, blandness of taste, ease of administration and elegant distinctive appearance.

Tablets may be plain, film or sugar coated bisected, embossed, layered or sustained-release. They can be made in a variety of sizes, shapes and colors. Tablets may be swallowed, chewed or dissolved in the buccal cavity or beneath the tongue. They may be dissolved in water for local or topical application. Sterile tablets are normally used for parenteral solutions and for implantation beneath the skin.

In addition to the active or therapeutic ingredients, tablets may contain a number of inert materials known as excipients. They may be classified according to the role they play in the final tablet. The primary composition includes a filler, binder, lubricant and glidant. Other excipients which give physical characteristics to the finished tablet are coloring agents, and flavors in the case of chewable tablets. Without excipients most drugs and pharmaceutical ingredients cannot be directly-compressed into tablets. This is primarily due to the poor flow and cohesive properties of most drugs. Typically, excipients are added to a formulation to impart good flow and compression characteristics to the material being compressed. Such properties are imparted to these excipients through pretreatment steps, such as wet granulation, slugging, spray drying spheronization or crystallization.

Lubricants are typically added to prevent the tableting materials from sticking to punches, minimize friction during tablet compression, and allow for removal of the compressed tablet from the die. Such lubricants are commonly included in the final tablet mix in amounts usually less than 1% by weight.

In addition, tablets often contain diluents which are added to increase the bulk weight of the blend resulting in a practical size for compression. This is often necessary where the dose of the drug is relatively small.

Another commonly used class of excipients in tablets is binders. Binders are agents, which impart cohesive qualities to the powdered material. Commonly used binders include starch, and sugars, such as sucrose, glucose, dextrose and lactose.

Disintegrants are often included to ensure that the tablet has an acceptable rate of disintegration. Typical disintegrants include starch derivatives and salts of carboxymethylcellulose.

Other desirable characteristics of excipients include the following:
High-compressibility to allow strong tablets to be made at low compression forces;
Good flow properties that can improve the flow of other excipients in the formula; and
Cohesiveness (to prevent tablet from crumbling during processing, shipping and handling).

There are three commercially important processes for making compressed tablets: wet granulation, direct compression and dry granulation (slugging or roller compaction). The method of preparation and type of excipients are selected to give the tablet formulation the desired physical characteristics that allow for the rapid compression of the tablets. After compression, the tablets must have a number of additional attributes, such as appearance, hardness, disintegrating ability and an acceptable dissolution profile. Choice of fillers and other excipients will depend on the chemical and physical properties of the drug, behavior of the mixture during processing and the properties of the final tablets. Preformulation studies are done to determine the chemical and physical compatibility of the active component with proposed excipients.

The properties of the drug, its dosage forms and the economics of the operation will determine selection of the best process for tableting. Generally, both wet granulation and direct compression are used in developing a tablet.

The dry granulation method may be used where one of the constituents, either the drug or the diluent, has sufficient cohesive properties to be tabletted. The method consists of blending, slugging the ingredients, dry screening, lubrication and compression.

The wet granulation method is used to convert a powder mixture into granules having suitable flow and cohesive properties for tableting. The procedure consists of mixing the powders in a suitable blender followed by adding the granulating solution under shear to the mixed powders to obtain a granulation. The damp mass is then screened through a suitable screen and dried by tray drying or fluidized bed drying. Alternately, the wet mass may be dried and passed through a mill. The overall process includes weighing, dry powder blending, wet granulating, drying, milling, blending lubrication and compression.

In general, powders do not have sufficient adhesive or cohesive properties to form hard, strong granules. A binder is usually required to bond the powder particles together due to the poor cohesive properties of most powders. Heat and moisture sensitive drugs cannot usually be manufactured using wet granulation. The large number of processing steps and processing time are problems due to high level manufacturing costs. Wet granulation has also been known to reduce the compressibility of some pharmaceutical excipients, such as microcrystalline cellulose.

Direct compression is regarded as a relatively quick process where the powdered materials are compressed directly without changing the physical and chemical properties of the drug. The active ingredient(s), direct compression excipients and other auxiliary substances, such as a glidant and lubricant are blended in a twin shell blender or similar low shear apparatus before being compressed into tablets. This type of mixing was believed to be essential in order to prepare “pharmaceutically acceptable” dosage forms. Some pharmaceutical scientists believe that the manner in which a lubricant is added to a formulation must be carefully controlled. Accordingly, lubricants are usually added to a granulation by gentle mixing. It is also believed that prolonged blending of a lubricant with a granulation can materially affect hardness and disintegration time for the resulting tablets. Excessive blending of lubricants with the granulate ingredients can cause water proofing of the granule and reduces tablet hardness or strength of the compressed tablet. For these reasons, high-shear mixing conditions have not been used to prepare direct compression dosage forms.

The advantages of direct compression include uniformity of blend, few manufacturing steps involved, i.e., the overall process involves weighing of powders, blending and compression, hence less cost; elimination of heat and moisture, prime particle dissociation and physical stability.

Pharmaceutical manufacturers would prefer to use direct compression techniques over wet or dry granulation methods because of quick processing time and cost advantages. However, direct compression is usually limited to those situations where the drug or active ingredient has physical characteristics required to form pharmaceutically acceptable tablets. However, one or more excipients must often be combined with the active ingredient before the direct-compression method can be used since many ingredients do not have the necessary properties. Since each excipient added to the formulation increases the tablet size of the final product, manufacturers are often limited to using the direct-compression method in formulations containing a low dose of the active ingredient per compressed tablet.

A solid dosage form containing a high-dose drug, i.e., the drug itself comprises a substantial portion of the total compressed tablet weight, could only be directly compressed if the drug itself has sufficient physical characteristics, e.g., cohesiveness, for the ingredients to be directly compressed.

For an example, the DPP-IV inhibitor e.g. those of formula (I) is considered a high-dose drug. Most tablet formulations include a range of 70-85% by weight of DPP-IV inhibitor per tablet. This high-dose drug, combined with its rather poor physical characteristics for direct compression, has not permitted direct compression as a method to prepare the final tablet. In addition, the active ingredients have poor stability in presence of water, another factor militating against the use of the wet granulation method.

Another limitation of direct compression as a method of tablet manufacturing is the potential size of the compressed tablets. If the amount of active ingredient is high, a pharmaceutical formulator may choose to wet granulate the active ingredient with other excipients to attain an acceptable sized tablet with the desired amount of active ingredient. The amount of filler, binder or other excipients needed in wet granulation is less than that required for direct compression since the process of wet granulation contributes toward the desired physical properties of the tablet.

Hydroxypropyl methylcellulose has been utilized in the pharmaceutical industry as a direct compression excipient for solid dose forms. Hydroxypropyl methylcellulose is a processed cellulose and controls drug release from solid dosage forms.

Despite the advantages of the direct compression, such as reduced processing time and cost, wet granulation is widely-used in the industry to prepare solid dosage forms. Wet granulation is often preferred over direct compression because wet granulation has a greater chance of overcoming any problems associated with the physical characteristics of various ingredients in the formulation. This provides material which has the required flow and cohesive properties necessary to obtain an acceptable solid dosage form.

The popularity of wet granulation compared to direct compression is based on at least three advantages. First, wet granulation provides the material to be compressed with better wetting properties, particularly in the case of hydrophobic drug substances. The addition of hydrophilic excipients makes the surface of the hydrophobic drug more hydrophilic, reducing disintegration and dissolution problems. Second, the content uniformity of the solid dosage form is generally improved with wet granulation because all of the granules usually contain the same amount of drug. Lastly, the segregation of drug(s) from excipients is avoided.

Segregation could be a potential problem with direct compression. The size and shape of particles comprising the granulate to be compressed are optimized through the wet granulation process. This is because when a dry solid is wet granulated the binder “glues” particles together, so that they agglomerate into spherical granules.

In spite of the advantages afforded by wet granulation in general, due to the instability of the compounds in the presence of water, it is desirable to directly compress tablets containing high-dose DPP-IV inhibitor, e.g. as that defined in formula (I). There is a need in the industry for techniques and pharmaceutical excipients which will allow manufacturers to prepare high-dose DPP-IV inhibitor tablets by direct compression.

It is an object of the invention to provide a DPP-IV inhibitor formulation in the form of a free-flowing, cohesive tableting powder, capable of being directly compressed into a tablet.

It is a further object of the invention to provide a direct compressed DPP-IV inhibitor tablet in unit dosage form having an acceptable dissolution profile, as well as acceptable degrees of hardness and resistance to chipping, as well as a short disintegration time.

It is a further object of the invention to provide a process for preparing a compressed DPP-IV inhibitor tablet by direct compression in unit dosage form.

The present invention provides a direct tableting, free-flowing particulate DPP-IV inhibitor formulation in the form of a tableting powder, capable of being directly compressed into a tablet having adequate hardness, rapid disintegration time and an acceptable dissolution pattern.

In addition to the active ingredient, the tableting powder contains a number of inert materials known as excipients. They may be classified according to the role they play in the final tablet. The primary composition includes fillers, binders or diluents, lubricants, disintegrants and glidants. Other excipients which give physical characteristics to the finished tablet are coloring agents, and flavors in the case of chewable tablets. Typically, excipients are added to a formulation to impart good flow and compression characteristics to the material being compressed.

The preferred formulation of this invention comprises the following: the active ingredient which is the DPP-IV inhibitor compound, the binders or diluents which are microcrystalline cellulose and lactose, the disintegrant which is sodium starch glycolate and the lubricant which is magnesium stearate.

One, two, three or more diluents can be selected. Examples of pharmaceutically acceptable fillers and pharmaceutically acceptable diluents include, but are not limited to, confectioner's sugar, compressible sugar, dextrates, dextrin, dextrose, lactose, mannitol, microcrystalline cellulose, powdered cellulose, sorbitol, sucrose and talc. The filler and/or diluent, e.g., may be present in an amount from about 15% to about 40% by weight of the composition. The preferred diluents include microcrystalline cellulose which is manufactured by the controlled hydrolysis of alpha-cellulose, obtained as a pulp from fibrous plant materials, with dilute mineral acid solutions. Following hydrolysis, the hydrocellulose is purified by filtration and the aqueous slurry is spray dried to form dry, porous particles of a broad size distribution. Suitable microcrystalline cellulose will have an average particle size of from about 20 nm to about 200 nm. Microcrystalline cellulose is available from several suppliers. Suitable microcrystalline cellulose includes Avicel PH 101, Avicel PH 102, Avicel PH 103, Avicel PH 105 and Avicel PH 200, manufactured by FMC Corporation. Particularly preferred in the practice of this invention is Avicel PH 102, which has the smallest surface area and pore structure. Preferably the microcrystalline cellulose is present in a tablet formulation in an amount of from about 25% to about 70% by weight. Another preferred range of this material is from about 30% to about 35% by weight; yet another preferred range of from about 30% to about 32% by weight.

Another diluent is lactose. Preferably, the lactose is ground to have an average particle size of between about 50 μm and about 500 μm prior to formulating. The lactose is present in the tablet formulation in an amount of from about 5% to about 40% by weight, and can be from about 18% to about 35% by weight, and most preferred, can be from about 20% to about 25% by weight.

One, two, three or more disintegrants can be selected. Examples of pharmaceutically acceptable disintegrants include, but are not limited to, starches; clays; celluloses; alginates; gums; cross-linked polymers, e.g., cross-linked polyvinyl pyrrolidone, cross-linked calcium carboxymethylcellulose and cross-linked sodium carboxymethylcellulose; soy polysaccharides; and guar gum. The disintegrant, e.g., may be present in an amount from about 2% to about 20%, e.g., from about 5% to about 10%, e.g., about 7% about by weight of the composition. A disintegrant is also an optional but useful component of the tablet formulation. Disintegrants are included to ensure that the tablet has an acceptable rate of disintegration. Typical disintegrants include starch derivatives and salts of carboxymethylcellulose. Sodium starch glycolate is the preferred disintegrant for this formulation. Preferably the disintegrant is present in the tablet formulation in an amount of from about 0% to about 10% by weight, and can be from about 1% to about 4% by weight, and most preferred, can be from about 1.5% to about 2.5% by weight.

One, two, three or more lubricants can be selected. Examples of pharmaceutically acceptable lubricants and pharmaceutically acceptable glidants include, but are not limited to, colloidal silica, magnesium trisilicate, starches, talc, tribasic calcium phosphate, magnesium stearate, aluminum stearate, calcium stearate, magnesium carbonate, magnesium oxide, polyethylene glycol, powdered cellulose and microcrystalline cellulose. The lubricant, e.g., may be present in an amount from about 0.1% to about 5% by weight of the composition; whereas, the glidant, e.g., may be present in an amount from about 0.1% to about 10% by weight. Lubricants are typically added to prevent the tableting materials from sticking to punches, minimize friction during tablet compression and allow for removal of the compressed tablet from the die. Such lubricants are commonly included in the final tablet mix in amounts usually less than 1% by weight. The lubricant component may be hydrophobic or hydrophilic. Examples of such lubricants include stearic acid, talc and magnesium stearate. Magnesium stearate reduces the friction between the die wall and tablet mix during the compression and ejection of the tablets. It helps prevent adhesion of tablets to the punches and dies. Magnesium stearate also aids in the flow of the powder in the hopper and into the die. It has a particle size range of 450-550 microns and a density range of 1.00-1.80 g/mL. It is stable and does not polymerize within the tableting mix. The preferred lubricant, magnesium stearate is also employed in the formulation. Preferably, the lubricant is present in the tablet formulation in an amount of from about 0.25% to about 6%; also preferred is a level of about 0.5% to about 4% by weight; and most preferably from about 0.1% to about 2% by weight. Other possible lubricants include talc, polyethylene glycol, silica and hardened vegetable oils. In an optional embodiment of the invention, the lubricant is not present in the formulation, but is sprayed onto the dies or the punches rather than being added directly to the formulation.

Other conventional solid fillers or carriers, such as, cornstarch, calcium phosphate, calcium sulfate, calcium stearate, magnesium stearate, stearic acid, glyceryl mono- and distearate, sorbitol, mannitol, gelatin, natural or synthetic gums, such as carboxymethyl cellulose, methyl cellulose, alginate, dextran, acacia gum, karaya gum, locust bean gum, tragacanth and the like, diluents, binders, lubricants, disintegrators, coloring and flavoring agents could optionally be employed.

Examples of pharmaceutically acceptable binders include, but are not limited to, starches; celluloses and derivatives thereof, e.g., microcrystalline cellulose, hydroxypropyl cellulose hydroxylethyl cellulose and hydroxylpropylmethyl cellulose; sucrose; dextrose; corn syrup; polysaccharides; and gelatin. The binder, e.g., may be present in an amount from about 10% to about 40% by weight of the composition.

Additional examples of useful excipients are described in the Handbook of pharmaceutical excipients, 3rd edition, Edited by A. H. Kibbe, Published by: American Pharmaceutical Association, Washington D.C., ISBN: 0-917330-96-X, or Handbook of Pharmaceutical Excipients (4^{th} edition), Edited by Raymond C Rowe—Publisher: Science and Practice which are incorporated herewith by reference.

Thus, in a first embodiment, the present invention concerns a pharmaceutical composition comprising;
(a) a DPP-IV inhibitor in free form or in acid addition salt form, preferably LAF237;
(b) a pharmaceutically acceptable diluent,
   wherein in the unit dosage form, the weight of DPP-IV inhibitor preferably LAF237 on a dry weight basis to tablet weight of diluent ratio is of 0.5 to 0.25, preferably 0.4 to 0.28.

In other words, the present invention concerns a pharmaceutical composition comprising;
(a) a DPP-IV inhibitor in free form or in acid addition salt form, preferably LAF237;
(b) a pharmaceutically acceptable diluent,
   wherein in the unit dosage form, the ratio of the weight of DPP-IV inhibitor preferably LAF237 to the weight of diluent is of 0.5 to 0.25, preferably 0.4 to 0.28.

Composition as described above, wherein at least one diluent is a microcrystalline cellulose and wherein in the unit dosage form, the weight of DPP-IV inhibitor preferably LAF237 on a dry weight basis to tablet weight of microcrystalline cellulose ratio is of 2 to 0.333, preferably 1 to 0.333, most preferably of 0.7 to 0.333. In other words, composition as described above, wherein at least one, diluent is a microcrystalline cellulose and wherein in the unit dosage form, the ratio of the weight of DPP-IV inhibitor preferably LAF237 to the weight of microcrystalline cellulose is of 2 to 0.333, preferably 1 to 0.333, most preferably of 0.7 to 0.333.

Composition as described above comprising between 20 and 120 mg of LAF237 preferably between 25 and 100 m of LAF237 or a pharmaceutically acceptable acid addition salt thereof.

Composition as described above wherein the diluent is selected from a microcrystalline cellulose and lactose, preferably microcrystalline cellulose and lactose are in the composition.

Composition as described above which comprises in addition;

(c) 0-20% by weight on a dry weight basis of a pharmaceutically acceptable disintegrant;
(d) 0.1-10% by weight on a dry weight basis of a pharmaceutically acceptable lubricant.

Preferably composition as described above which comprises in addition;

(c) 1-6% by weight on a dry weight basis of a pharmaceutically acceptable disintegrant;
(d) 0.25-6% by weight on a dry weight basis of a pharmaceutically acceptable lubricant.

The above ratios have been obtained on a dry weight basis for the DPP-IV inhibitors and diluents.

The unit dosage form, is any kind of pharmaceutical dosage form such as capsules, tablets, granules, chewable tablets, etc.

In a further embodiment, the present invention concerns a pharmaceutical composition comprising;
(a) 5-60% by weight on a dry weight basis of a DPP-IV inhibitor in free form or in acid addition salt form, preferably LAF237;
(b) 40-95% by weight on a dry weight basis of a pharmaceutically acceptable diluent;
(c) 0-20% by weight on a dry weight basis of a pharmaceutically acceptable disintegrant; and optionally
(d) 0.1-10% by weight on a dry weight basis of a pharmaceutically acceptable lubricant.

Preferably the present invention concerns a pharmaceutical composition comprising;
(a) 20-40% by weight on a dry weight basis of a DPP-IV inhibitor in free form or in acid addition salt form, preferably LAF237;
(b) 40-95% by weight on a dry weight basis of a pharmaceutically acceptable diluent;
(c) 0-10% by weight on a dry weight basis of a pharmaceutically acceptable disintegrant; and optionally
(d) 0.25-6% by weight on a dry weight basis of a pharmaceutically acceptable lubricant.

Preferably the present invention concerns a pharmaceutical composition comprising;
(a) 20-40% by weight on a dry weight basis of a DPP-IV inhibitor in free form or in acid addition salt form, preferably LAF237;
(b) 40-80% by weight on a dry weight basis of a pharmaceutically acceptable diluent;
(c) 0-10% by weight on a dry weight basis of a pharmaceutically acceptable disintegrant; and optionally
(d) 0.25-6% by weight on a dry weight basis of a pharmaceutically acceptable lubricant.

Most preferably the present invention concerns a pharmaceutical composition comprising;
(a) 20-35% by weight on a dry weight basis of a DPP-IV inhibitor in free form or in acid addition salt form, preferably LAF237;
(b) 40-95% by weight on a dry weight basis of a pharmaceutically acceptable diluent;
(c) 0-10% by weight on a dry weight basis of a pharmaceutically acceptable disintegrant; and optionally
(d) 0.25-6% by weight on a dry weight basis of a pharmaceutically acceptable lubricant.

Most preferably the present invention concerns a pharmaceutical composition comprising;
(a) 20-35% by weight on a dry weight basis of a DPP-IV inhibitor in free form or in acid addition salt form, preferably LAF237;
(b) 62-78% by weight on a dry weight basis of a pharmaceutically acceptable diluent;
(c) 0-10% by weight on a dry weight basis of a pharmaceutically acceptable disintegrant; and optionally
(d) 0.1-10% by weight on a dry weight basis of a pharmaceutically acceptable lubricant.

Most preferably the present invention concerns a pharmaceutical composition comprising;
(a) 20-35% by weight on a dry weight basis of a DPP-IV inhibitor in free form or in acid addition salt form, preferably LAF237;
(b) 62-78% by weight on a dry weight basis of a pharmaceutically acceptable diluent;
(c) 1-6% by weight on a dry weight basis of a pharmaceutically acceptable disintegrant; and optionally
(d) 0.25-6% by weight on a dry weight basis of a pharmaceutically acceptable lubricant.

Most preferably the present invention concerns a pharmaceutical composition comprising;
(a) 22-28% by weight on a dry weight basis of a DPP-IV inhibitor in free form or in acid addition salt form, preferably LAF237;
(b) 66-76% by weight on a dry weight basis of a pharmaceutically acceptable diluent;
(c) 0-6% by weight on a dry weight basis of a pharmaceutically acceptable disintegrant; and optionally
(d) 0.25-6% by weight on a dry weight basis of a pharmaceutically acceptable lubricant.

Most preferably the present invention concerns a pharmaceutical composition comprising;
(a) 22-28% by weight on a dry weight basis of a DPP-IV inhibitor in free form or in acid addition salt form, preferably LAF237;
(b) 66-76% by weight on a dry weight basis of a pharmaceutically acceptable diluent;
(c) 1-6% by weight on a dry weight basis of a pharmaceutically acceptable disintegrant; and optionally
(d) 0.25-6% by weight on a dry weight basis of a pharmaceutically acceptable lubricant.

Composition as described above which comprises in addition;

(c) 1-6% by weight on a dry weight basis of a pharmaceutically acceptable disintegrant;
(d) 0.1-10% by weight on a dry weight basis of a pharmaceutically acceptable lubricant.

In the present application the reference to “a pharmaceutically acceptable diluent” means at least one diluent, a mixture of e.g. 2 or 3 diluents is also covered.

Preferably the above described compositions comprise;
i) one or two diluents selected from microcrystalline cellulose and lactose
ii) the two diluents microcrystalline cellulose and lactose,
iii) 25-70% preferably 35-55% by weight on a dry weight basis of a pharmaceutically acceptable microcrystalline cellulose, or
iv) 25-70% preferably 35-55% by weight on a dry weight basis of a pharmaceutically acceptable microcrystalline cellulose and 5-40% preferably 18-35% of lactose.

Most preferably the above described compositions comprise one or two diluents selected from microcrystalline cellulose such as Avicel PH 102 and lactose.

Most preferably the pharmaceutical composition comprises the pharmaceutically acceptable lubricant (d).

In the present application the reference to a pharmaceutically acceptable disintegrant means at least one disintegrant, a mixture of e.g. 2 or 3 disintegrants is also covered.

In the present application the reference to a pharmaceutically acceptable lubricant means at least one lubricant, a mixture of e.g. 2 or 3 lubricants is also covered.

Preferred DPP-IV inhibitor is LAF237, preferred diluents are microcrystalline cellulose or lactose or preferably a combination of microcrystalline cellulose and lactose, preferred disintegrant is sodium starch glycolate, and preferred lubricant is magnesium stearate.

The particular components in the preferred composition are the following:
(a) 20-35% by weight on a dry weight basis of DPP-IV inhibitor e.g. LAF237;
(b) 25-70% by weight on a dry weight basis of a pharmaceutically acceptable microcrystalline cellulose;
(c) 5-40% by weight on a dry weight basis of a pharmaceutically acceptable lactose;
(d) 0-10% by weight on a dry weight basis of a pharmaceutically acceptable sodium starch glycolate;
(e) 0.25-6% by weight on a dry weight basis of magnesium stearate.

The particular components in the preferred composition are the following:
(a) 25-35% by weight on a dry weight basis of DPP-IV inhibitor e.g. LAF237;
(b) 25-70% by weight on a dry weight basis of a pharmaceutically acceptable microcrystalline cellulose;
(c) 5-40% by weight on a dry weight basis of a pharmaceutically acceptable lactose;
(d) 0-10% by weight on a dry weight basis of a pharmaceutically acceptable sodium starch glycolate;
(e) 0.25-6% by weight on a dry weight basis of magnesium stearate.

Another preferred composition is the following:
(a) from about 30% to about 32% by weight on a dry weight basis of a DPP-IV inhibitor or a DPP-IV inhibitor of formula (I);
(b) from about 40% to about 45% by weight on a dry weight basis of a pharmaceutically acceptable microcrystalline cellulose;
(c) from about 20% to about 25% by weight on a dry weight basis of a pharmaceutically acceptable lactose;
(d) from about 1.5% to about 2.5% by weight on a dry weight basis of a pharmaceutically acceptable sodium starch glycolate; and
(e) from about 0.1% to about 2% by weight on a dry weight basis of magnesium stearate.

Another preferred composition is the following:
(a) 20-35% preferably 22-28% by weight on a dry weight basis of DPP-IV inhibitor e.g. LAF237;
(b) 35-55% by weight on a dry weight basis of a pharmaceutically acceptable microcrystalline cellulose;
(c) 18-35% by weight on a dry weight basis of a pharmaceutically acceptable lactose;
(d) 1-4% by weight on a dry weight basis of a pharmaceutically acceptable sodium starch glycolate; and
(e) 0.5-4% by weight on a dry weight basis of magnesium stearate.

Still another preferred composition is the following:
(a) from about 22% to about 28% preferably 24-26% by weight on a dry weight basis of a DPP-IV inhibitor or a DPP-IV inhibitor of formula (I);
(b) from about 45% to about 50% by weight on a dry weight basis of a pharmaceutically acceptable microcrystalline cellulose;
(c) from about 20% to about 25% by weight on a dry weight basis of a pharmaceutically acceptable lactose;
(d) from about 1.5% to about 2.5% by weight on a dry weight basis of a pharmaceutically acceptable sodium starch glycolate; and
(e) from about 0.1% to about 2% by weight on a dry weight basis of magnesium stearate.

Still another preferred composition is the following:
(a) from 24-26% by weight on a dry weight basis of a DPP-IV inhibitor or a DPP-IV inhibitor of formula (I);
(b) from about 46% to about 48% by weight on a dry weight basis of a pharmaceutically acceptable microcrystalline cellulose;
(c) from about 23% to about 24.5% by weight on a dry weight basis of a pharmaceutically acceptable lactose;
(d) from about 1.5% to about 2.5% by weight on a dry weight basis of a pharmaceutically acceptable sodium starch glycolate; and
(e) from about 0.1% to about 2% by weight on a dry weight basis of magnesium stearate.

Still another preferred composition is the following:
(a) 30-35% by weight on a dry weight basis of DPP-IV inhibitor e.g. LAF237;
(b) 35-50% by weight on a dry weight basis of a pharmaceutically acceptable microcrystalline cellulose;
(c) 18-35% by weight on a dry weight basis of a pharmaceutically acceptable lactose;
(d) 1-4% by weight on a dry weight basis of a pharmaceutically acceptable sodium starch glycolate; and
(e) 0.5-4% by weight on a dry weight basis of magnesium stearate.

In a further embodiment, the present invention concerns any one of the above described compositions wherein the pharmaceutically acceptable lubricant (d) is only optionally comprised in the formulation. But preferably the pharmaceutically acceptable lubricant (d) is comprised in the composition.

Preferably for compressed tablets especially for direct compressed tablets, the above described compositions comprise between 20 and 35% most preferably between 22 and 28% by weight on a dry weight basis of a DPP-IV inhibitor especially LAF237, in free form or in acid addition salt form.

In the present application the terms composition and formulation have the same meaning.

Additional conventional excipients can optionally be added to the herein described formulations such as the conventional solid fillers or carriers described hereinabove.

The above described composition are particularly adapted for the production of pharmaceutical tablets e.g compressed tablets or preferably direct compressed tablets, caplets or capsules and provides the necessary physical characteristics, dissolution and drug release profiles as required by one of ordinary necessary physical skill in the art. Therefore in an additional embodiment, the present invention concerns the use of any of the above described formulations, for the manufacture of pharmaceutical tablets, caplets or capsules in particular for granulation, direct compression and dry granulation (slugging or roller compaction).

The above composition are also particularly useful for the production of tablets especially compressed tablets and very preferably direct compressed tablets.

In particular the tablets obtained with the above described formulations especially when processed in the form of direct compressed tablets or the below described direct compressed tablets, have very low friability problems, very good breaking strength, improved manufacturing robustness, optimal tablet thickness to tablet weight ratios (direct compressed tablets), less water in the formulation especially directed compressed tablet, good Dispersion Disintegration time DT according to the British Pharmacopoeia 1988, good Dispersion Quality.

This present invention of direct compression of DPP-IV inhibitor involves blending and compression. The choice of grades of excipients took into consideration particle size maintained within a range that allows homogeneity of the powder mix and content uniformity of DPP-IV inhibitor. It prevents segregation of powders in the hopper during direct compression. The advantages of using these excipients are that they impart compressibility, cohesiveness and flowability of the powder blend. In addition, the use of direct compression provides competitive unit production cost, shelf life, eliminates heat and moisture, allows for prime particle dissociation, physical stability and ensures particle size uniformity.

The described advantages of the claimed compositions are also very useful for e.g. roller compaction or wet granulation or to fill capsules.

In the development of the herein described pharmaceutical compositions, the applicant has discovered that the compressed tablets especially direct compressed tablet is particularly advantageous if;
i) the particles comprising the DPP-IV inhibitor have a particle size distribution between less than 250 μm, preferably between 10 to 250 μm, and/or
ii) the water content of the tablet at less than 10% after 1 week at 25° C. and 60% room humidity (RH), and/or
iii) tablet thickness to tablet weight ratios is of 0.002 to 0.06 mm/mg.

The present invention concerns a compressed pharmaceutical tablet preferably a direct compressed tablet, comprising a DPP-IV inhibitor, in free form or in acid addition salt form, said DPP-IV inhibitor having physical properties that render the tableting into compressed preferably direct compressed pharmaceutical tablet unlikely or very difficult. Preferred DPP-IV inhibitor is LAF237. The physical properties can be e.g. bulkiness, fluffiness and the like. During the further development of the herein described pharmaceutical compositions, the applicant has discovered that the processing properties or physical properties of the formulation, such as hydroscopicity, flowability, bulkiness, fluffiness is surprisingly improved if the particles comprising the DPP-IV inhibitor have a particle size distribution between less than 250 μm, or between 10 to 250 μm or between 50 to 150 μm. The applicant also surprisingly discovered that the tablets show improved physical characteristics such as solubility, friability, hydroscopicity, hardness etc, if at least one of the above described criteria i), ii) and/or iii) is respected.

Thus in a first embodiment (a), the present invention concerns compressed tablets preferably direct compressed pharmaceutical tablets, wherein the dispersion contains particles comprising DPP-IV inhibitor preferably LAF237, in free form or in acid addition salt form, and wherein at least 40%, preferably 60%, most preferably 80% even more preferably 90% of the particle size distribution in the tablet is less than 250 μm or preferably between 10 to 250 μm.

The present invention concerns compressed tablets preferably direct compressed pharmaceutical tablets, wherein the dispersion contains particles comprising DPP-IV inhibitor preferably LAF237, in free form or in acid addition salt form, and wherein at least 40%, preferably 60%, most preferably 80% even more preferably 90% of the particle size distribution in the tablet is greater than 10 μm.

The term “wherein at least 40%, preferably 60%, most preferably 80% even more preferably 90%” means at least 40%, preferably at least 60%, most preferably at least 80%, even more preferably at least 90%.

The term “wherein at least at least 25%, preferably 35% and most preferably 45%” means at least 25%, preferably at least 35% and most preferably at least 45%.

In particular the present invention concerns compressed tablets preferably direct compressed pharmaceutical tablets, wherein the dispersion contains particles comprising DPP-IV inhibitor preferably LAF237, in free form or in acid addition salt form, and wherein at least 25%, preferably 35% and most preferably 45% of the particle size distribution in the tablet is between 50 to 150 μm.

In a second embodiment (b), this invention concerns a compressed tablet, preferably a direct compressed pharmaceutical tablet wherein the dispersion contains particles comprising DPP-IV inhibitor preferably LAF237, in free form or in acid addition salt form, and wherein tablet thickness to tablet weight ratios is of 0.002 to 0.06 mm/mg preferably of 0.01 to 0.03 mm/mg.

The combination of the above first and second embodiments (a) and (b), provide compressed tablets preferably direct compressed tablets with good compaction characteristics.

Thus this invention concerns also a compressed tablet, preferably a direct compressed tablet wherein the dispersion contains particles comprising DPP-IV inhibitor preferably LAF237, in free form or in acid addition salt form, and wherein;
i) at least 40%, preferably 60%, most preferably 80% even more preferably 90% of the particle size distribution in the tablet is between 10 to 250 μm, and
ii) tablet thickness to tablet weight ratios is of 0.002 to 0.06 mm/mg or of 0.01 to 0.03 mm/mg
   preferably wherein;
i) at least 25%, preferably 35% and most preferably 45% of the particle size distribution in the tablet is between 50 to 150 μm, and
ii) tablet thickness to tablet weight ratios is of 0.002 to 0.06 mm/mg or of 0.01 to 0.03 mm/mg.

In a third embodiment, this invention concerns a compressed tablet preferably a direct compressed pharmaceutical tablet wherein the dispersion contains particles comprising DPP-IV inhibitor preferably LAF237, in free form or in acid addition salt form, and wherein;
i) at least 40%, preferably 60%, most preferably 80% even more preferably 90% of the particle size distribution in the tablet is between 10 to 250 μm,
ii) the water content of the tablet is less than 10% after 1 week at 25° C. and 60% RH, and
iii) tablet thickness to tablet weight ratios is of 0.002 to 0.06 mm/mg.

Preferably this invention concerns a compressed tablet most preferably a direct compressed tablet wherein the dispersion contains particles comprising DPP-IV inhibitor preferably LAF237, in free form or in acid addition salt form, and wherein;
i) at least 25%, preferably 35% and most preferably 45% of the particle size distribution in the tablet is between 50 to 150 μl,
ii) the water content of the tablet is less than 10% after 1 week at 25° C. and 60% RH, and
iii) tablet thickness to tablet weight ratios is of 0.002 to 0.06 mm/mg.

Preferably this invention concerns a compressed tablet most preferably a direct compressed tablet wherein the dispersion contains particles comprising DPP-IV inhibitor preferably LAF237, in free form or in acid addition salt form, and wherein;
i) at least 25%, preferably 35% and most preferably 45% of the particle size distribution in the tablet is between 50 to 150 μm,
ii) the water content of the tablet is less than 5% after 1 week at 25° C. and 60% RH, and
iii) tablet thickness to tablet weight ratios is of 0.002 to 0.06 mm/mg.

Preferably this invention concerns a compressed tablet, most preferably a direct compressed tablet wherein the dispersion contains particles comprising DPP-IV inhibitor preferably LAF237, in free form or in acid addition salt form, and wherein;
i) at least 25%, preferably 35% and most preferably 45% of the particle size distribution in the tablet is between 50 to 150 μm,
ii) the water content of the tablet is less than 5% after 1 week at 25° C. and 60% RH, and
iii) tablet thickness to tablet weight ratios is of 0.01 to 0.03 mm/mg

In a very preferred aspect, the above described three embodiments i.e. compressed tablets and direct compressed tablets contain the herein described compositions such as a pharmaceutical composition comprising;
(a) 5-60% by weight on a dry weight basis of a DPP-IV inhibitor in free form or in acid addition salt form, preferably LAF237;
(b) 40-95% or 40-80% by weight on a dry weight basis of a pharmaceutically acceptable diluent;
(c) 0-20% by weight on a dry weight basis of a pharmaceutically acceptable disintegrant; and optionally
(d) 0.1-10% by weight on a dry weight basis of a pharmaceutically acceptable lubricant, or
   a pharmaceutical composition comprising;
(a) 20-35% by weight on a dry weight basis of a DPP-IV inhibitor in free form or in acid addition salt form, preferably LAF237;
(b) 40-95% or 40-80%, preferably 62-78% by weight on a dry weight basis of a pharmaceutically acceptable diluent;
(c) 0-10% by weight on a dry weight basis of a pharmaceutically acceptable disintegrant;
(d) 0.25-6% by weight on a dry weight basis of a pharmaceutically acceptable lubricant.

Preferably the DPPIV particles especially the LAF237 particles comprise more than 60% of DPPIV inhibitor, most preferably more than 90% or 95% and even more preferably more than 98% of DPPIV inhibitor. DPPIV particles especially the LAF237 particles can alternatively be formed by microgranulation, process well known in the art, and contain up to 40% of a pharmaceutically acceptable excipient.

Preferably the LAF237 particles comprise more than 60% of LAF237, most preferably more than 90% or 95% and even more preferably more than 98% of LAF237.

It has been discovered that the selected particle size distribution of DPPIV inhibitor especially LAF237 were particularly important to provide the best compaction of the tablets.

In an additional preferred embodiment, the particle size distribution of the selected excipients (b), (c) and/or (d) is similar to the particle size distribution of the DPP-IV inhibitor particles preferably LAF237 particles.

The term “similar”, means that the particle size distribution of the excipient in the tablet is between 5 and 400 μm, or between 10 and 300 μm, preferably between 10 to 250 μm.

The preferred excipients with an adapted particle size distribution can be picked from e.g. Handbook of Pharmaceutical Excipients (4^{th} edition), Edited by Raymond C Rowe—Publisher: Science and Practice.

Particle size of drug, e.g. LAF237 particles size, is controlled by crystallisazion, drying and/or milling/sieving (non limiting examples are described below). Particle size can also be comminuted using roller compaction and milling/sieving. Producing the right particle size is well known and described in the art such as in “Pharmaceutical dosage forms: volume 2, 2nd edition, Ed.: H. A. Lieberman, L. Lachman, J. B. Schwartz (Chapter 3: SIZE REDUCTION)”. The desired particle size distribution can be obtained from any form of the DPP-IV inhibitors especially from any form of LAF237 such as from amorphous LAF237, crystalline forms of LAF237, crystalline form “A” of LAF237, a partially crystalline form of LAF237, a polymorphous form of LAF237, a solvate form of LAF237 or an hydrate form of LAF237, or any salt thereof. Preferred particles are obtained from crystalline form “A” of LAF237.

Multiple particle sizes have been studied and it has been discovered that the herein described specific size range provides unexpected good results for direct compaction.

PARTICLE SIZE DISTRIBUTION ESTIMATION BY ANALYTICAL SIEVING: Particle size distribution is measured using Sieve analysis, Photon Correlation Spectroscopy or laser diffraction (international standart ISO 13320-1), or electronic sensing zone, light obstruction, sedimentation or microscopy which are procedures well known by the person skilled in the art. Sieving is one of the oldest methods of classifying powders by particle size distribution. Such methods are well known and described in the art such as in any analytical chemistry text book or by the United State Pharmacopeia's (USP) publication USP-NF (2004—Chapter 786—(The United States Pharmacopeial Convention, Inc., Rockville, Md.)) which describes the US Food and Drug Administration (FDA) enforceable standards. The used techniques are e.g. described in Pharmaceutical dosage forms: volume 2, 2nd edition, Ed.: H. A. Lieberman, L. Lachman, J. B. Schwartz is a good example. It also mentions (page 187) additional methods: Electronic sensing zone, light obstruction, air permeation, sedimentation in gas or liquid.

In an air jet sieve measurement of particle size, air is drawn upwards, through a sieve, from a rotating slit so that material on the sieve is fluidised. At the same time a negative pressure is applied to the bottom of the sieve which removes fine particles to a collecting device. Size analyses and determination of average particle size are performed by removal of particles from the fine end of the size distribution by using single sieves consecutively. See also “Particle Size Measurement”, 5th Ed., p 178, vol. 1; T. Allen, Chapman & Hall, London, UK, 1997, for more details on this. For a person skilled in the art, the size measurement as such is thus of conventional character.

Water content of the tablet can be measured using Loss on drying method or Karl-Fischer method which are well known methods to the person skilled in the art (e.g. water content can be measured by loss on drying by thermogrametry). Such methods are well known and described in the art such as in any analytical chemistry text book (J. A. Dean, Analytical Chemistry Handbook, Section 19, McGraw-Hill, New York, 1995) or by the United State Pharmacopeia's (USP) publication USP-NF (2004) which describes the US Food and Drug Administration (FDA) enforceable standards ((2004—USP—Chapter 921).

Tablet thickness is measurable using a ruler, vernier caliper, a screw gauge or any electronic method to measure dimensions. We take the tablet thickness in mm and divide by tablet weight in mg to get the ratio. Such methods are well known and described in the art such as in any analytical chemistry text book or by the United State Pharmacopeia's (USP) publication USP-NF (2004) which describes the US Food and Drug Administration (FDA) enforceable standards.

This invention provides in particular a compressed tablet or direct compressed tablet which is capable of dispersing in water within a period of 5 to 15 minutes to provide a dispersion which is capable of passing through a sieve screen with a mesh aperture of 710 μm in accordance with the herein defined British Pharmacopoeia test for dispersible tablets.

A tablet according to the invention, as well as being quickly dispersible in water, has the added advantage that it meets the British Pharmacopoeia (B.P.) test for dispersible tablets in respect of dispersion times and dispersion quality (i.e. passage through a 710 μm sieve).

Preferably the dispersion time of a tablet according to the invention is less than 15 minutes, more preferably less than 12 minutes and most preferably less than 10 minute.

A further advantage of the tablets according to invention is that because a relatively fine dispersion is formed the tablet will have a lower dissolution time and thus the drug may be absorbed into the blood stream much faster. Furthermore the fast dispersion times and relatively fine dispersions obtained with tablets according to the invention are also advantageous for swallowable tablets. Thus tablets according to the invention can be presented both for dispersion in water and also for directly swallowing. Those tablets according to the invention that are intended for swelling are preferably film-coated to aid swallowing.

In a further embodiment the present invention concerns a compressed tablet with improved dissolution rates (dissolution of the drug), wherein the dispersion contains particles i.e. DPPIV particles especially LAF237 particles comprising DPP-IV inhibitor preferably LAF237, in free form or in acid addition salt form, wherein at least 40%, preferably 60%, most preferably 80% even more preferably 90% of the particle size distribution in the tablet is between 10 to 250 mm,

and wherein
i) between 0 and 10 minutes 85 to 99.5% of the active ingredient is released, and
ii) between 10 and 15 minutes 90 to 99.5% of the active ingredient is released, preferably wherein,
i) between 0 and 10 minutes 88 to 99.5% of the active ingredient is released, and
ii) between 10 and 15 minutes 95 to 99.5% of the active ingredient is released,
or preferably
i) between 0 and 10 minutes 89 to 94% of the active ingredient is released, and
ii) between 10 and 15 minutes 96 to 99% of the active ingredient is released.

The Paddle method to measure the drug dissolution rate (% of release) is used with 1000 ml of 0.01N HCl. Such methods are well known and described in the art such as in any analytical chemistry text book or by the United State Pharmacopeia's (USP) publication USP-NF (2004—Chapter 711) which describes the US Food and Drug Administration (FDA) enforceable standards.

The present invention also concerns the use of a DPP-IV inhibitor especially vildagliptin for the preparation of a compressed or a directly compressed tablet, wherein at least 40%, preferably 60%, most preferably 80% even more preferably 90%, of the DPP-IV inhibitor especially vildagliptin has a particle size distribution of less than 250 μm or preferably between 10 to 250 μm.

In a further embodiment, the present invention concerns any one of the above described pharmaceutical compositions wherein the DPP-IV inhibitor especially vildagliptin or the vildagliptin crystal “Form A” has a particle size distribution as defined for above described compressed tablets.

Thus in a further embodiment, the invention also concerns a pharmaceutical composition such as described herein, wherein the dispersion contains particles (such as described hereinabove) comprising a DPP-IV inhibitor especially vildagliptin or a vildagliptin crystalline form or the crystal “Form A” of vildagliptin or a pharmaceutical salts thereof and wherein;
i) at least 40%, preferably 60%, of the particle size distribution in the formulation is less than 250 μm, and/or
ii) at least 40%, preferably 60%, of the particle size distribution in the formulation is between 10 to 250 μm, and/or
iii) at least 60%, preferably at least 80%, of the particle size distribution in the formulation is between 10 to 250 μm, and/or
iv) at least 25% or at least 35% of the particle size distribution in the formulation is between 50 to 150 μm.

In an additional embodiment the particle size distribution of the pharmaceutical excipients in the above formulation is between 5 and 400 μm.

The invention also provides a process for preparing a compressed or a directly compressed tablet comprising a DPP-IV inhibitor especially vildagliptin wherein at least 40%, preferably 60%, most preferably 80% even more preferably 90% of the DPP-IV inhibitor, especially vildagliptin used in the process has a particle size distribution of less than 250 μm or preferably between 10 to 250 μm.

The invention also provides a process for preparing a compressed DPP-IV inhibitor tablet preferably a directly compressed tablet, in unit dosage form;

which comprises:
(a) blending as a % by weight on a dry weight basis:
(i) 5-60% by weight on a dry weight basis of a DPP-IV inhibitor especially vildagliptin wherein at least 40%, preferably 60%, most preferably 80% even more preferably 90%, of the DPP-IV inhibitor especially vildagliptin has a particle size distribution of less than 250 μm or preferably between 10 to 250 μm; and
(ii) and at least one excipient selected from a diluent, a disintegrant and a lubricant,
   to form a DPP-IV inhibitor formulation in the form of a tableting powder, capable of being compressed preferably directly compressed into a tablet; and
   (b) compressing the formulation prepared during step (a) to form the compressed DPP-IV inhibitor tablet in unit dosage form.

The invention also provides a process for preparing a compressed DPP-IV inhibitor tablet preferably a directly compressed tablet, in unit dosage form, wherein;
i) tablet thickness to tablet weight ratios is of 0.002 to 0.06 mm
   which comprises:
   (a) blending as a % by weight on a dry weight basis:
(i) 5-60% by weight on a dry weight basis of DPP-IV inhibitor e.g. vildagliptin wherein at least 40%, preferably 60%, most preferably 80% even more preferably 90%, of the DPP-IV inhibitor especially vildagliptin has a particle size distribution of less than 250 μm or preferably between 10 to 250 μm; and
(ii) and at least one excipient selected from a diluent, a disintegrant and a lubricant,
   to form a DPP-IV inhibitor formulation in the form of a tableting powder, capable of being compressed preferably directly compressed into a tablet; and
   (b) compressing the formulation prepared during step (a) to form the compressed DPP-IV inhibitor tablet in unit dosage form.

The invention also provides a process for preparing a compressed DPP-IV inhibitor tablet preferably a directly compressed tablet, in unit dosage form, wherein;
i) at least 40%, preferably 60%, most preferably 80% even more preferably 90% of the particles comprising DPP-IV inhibitor preferably vildagliptin, in free form or in acid addition salt form, in the tablet have a particle size distribution of less than 250 μm preferably between 10 to 250 μm,
ii) the water content of the tablet is less than 10% after 1 week at 25° C. and 60% RH, and
iii) tablet thickness to tablet weight ratios is of 0.002 to 0.06 mm
   which comprises:
   (a) blending as a % by weight on a dry weight basis:
(i) 5-60% or 6-60% by weight on a dry weight basis of DPP-IV inhibitor e.g. vildagliptin wherein at least 40%, preferably 60%, most preferably 80% even more preferably 90%, of the DPP-IV inhibitor especially vildagliptin has a particle size distribution of less than 250 μm or preferably between 10 to 250 μm; and
(ii) and at least one excipient selected from a diluent, a disintegrant and a lubricant,
   to form a DPP-IV inhibitor formulation in the form of a tableting powder, capable of being compressed preferably directly compressed into a tablet; and
   (b) compressing the formulation prepared during step (a) to form the compressed DPP-IV inhibitor tablet in unit dosage form.

In a further preferred embodiment at least 25%, preferably 35% and most preferably 45% of the particle size distribution of the DPP-IV inhibitor especially vildagliptin used in the herein described process is between 50 to 150 μm.

Preferably the above described process comprises:

(a) blending as a % by weight on a dry weight basis:
(i) 5-60% by weight, on a dry weight basis of DPP-IV inhibitor e.g. LAF237 wherein at least 40%, preferably 60%, most preferably 80% even more preferably 90%, of the DPP-IV inhibitor especially vildagliptin has a particle size distribution of less than 250 μm or preferably between 10 to 250 μm;
(ii) 40-95% by weight on a dry weight basis of a pharmaceutically acceptable diluent;
(iii) 0-20% by weight on a dry weight basis of a pharmaceutically acceptable disintegrant; and
(iv) 0.1-10% by weight on a dry weight basis of a pharmaceutically acceptable lubricant,
   to form a DPP-IV inhibitor formulation in the form of a tableting powder, capable of being compressed preferably directly compressed into a tablet; and
   (b) compressing the formulation prepared during step (a) to form the compressed DPP-IV inhibitor tablet in unit dosage form.

Most preferably the process comprises:

(a) blending as a % by weight on a dry weight basis:
(i) 20-35% or 25-35% by weight on a dry weight basis of DPP-IV inhibitor e.g. LAF237 wherein at least 40%, preferably 60%, most preferably 80% even more preferably 90%, of the DPP-IV inhibitor especially vildagliptin has a particle size distribution of less than 250 μm or preferably between 10 to 250 μm;
(ii) 40-80% or 40-95% by weight on a dry weight basis of a pharmaceutically acceptable diluent;
(iii) 0-10% by weight on a dry weight basis of a pharmaceutically acceptable disintegrant; and
(iv) 0.25-6% by weight on a dry weight basis of a pharmaceutically acceptable lubricant,
   to form a DPP-IV inhibitor formulation in the form of a tableting powder, capable of being compressed preferably directly compressed into a tablet; and
   (b) compressing the formulation prepared during step (a) to form the compressed DPP-IV inhibitor tablet in unit dosage form.

Preferably the blended composition used in step (a) is selected from the herein described preferred formulations.

Preferred DPP-IV inhibitor is LAF237, preferred diluents are microcrystalline cellulose or lactose or preferably a combination of microcrystalline cellulose and lactose, preferred disintegrant is sodium starch glycolate, and preferred lubricant is magnesium stearate.

In a best embodiment the process comprises:

(a) blending as a % by weight on a dry weight basis:
(i) 20-35% or preferably 25-30% by weight by weight on a dry weight basis of DPP-IV inhibitor preferably vildagliptin, in free form or in acid addition salt form wherein at least 40%, preferably 60%, most preferably 80% even more preferably 90%, of the DPP-IV inhibitor especially vildagliptin has a particle size distribution of less than 250 μm or preferably between 10 to 250 μm;
(ii) 25-70% by weight or preferably 35-50% by weight on a dry weight basis of a pharmaceutically acceptable microcrystalline cellulose such as Avicel PH 102;
(iii) 5-40% by weight or preferably 18-35% by weight on a dry weight basis of a pharmaceutically acceptable lactose;
(iv) 0-10% by weight or preferably 1-4% by weight on a dry weight basis of a pharmaceutically acceptable sodium starch glycolate; and
(v) 0.25-6% by weight or preferably 0.5-4% by weight on a dry weight basis of a pharmaceutically acceptable magnesium stearate.
   to form a DPP-IV inhibitor formulation in the form of a tableting powder, capable of being compressed preferably directly compressed into a tablet; and
   (b) compressing the formulation prepared during step (a) to form the compressed DPP-IV inhibitor tablet in unit dosage form.

The invention also provides a process for preparing a compressed DPP-IV inhibitor tablet in unit dosage form which comprises:

(a) blending as a % by weight on a dry weight basis:
(i) 30-32% by weight on a dry weight basis of DPP-IV inhibitor preferably LAF237, in free form or in acid addition salt form wherein at least 40%, preferably 60%, most preferably 80% even more preferably 90%, of the DPP-IV inhibitor especially vildagliptin has a particle size distribution of less than 250 μm or preferably between 10 to 250 μm;
(ii) 40-45% by weight on a dry weight basis of a pharmaceutically acceptable microcrystalline cellulose (Avicel PH 102);
(iii) 20-25% by weight on a dry weight basis of a pharmaceutically acceptable lactose;
(iv) 1.5-2% by weight on a dry weight basis of a pharmaceutically acceptable sodium starch glycolate; and
(v) 0.1-2% by weight on a dry weight basis of magnesium stearate,
   to form a DPP-IV inhibitor formulation in the form of a tableting powder, capable of being compressed preferably directly compressed into a tablet; and
   (b) compressing the formulation prepared during step (a) to form the compressed DPP-IV inhibitor tablet in unit dosage form.

The invention also provides a process for preparing a compressed DPP-IV inhibitor tablet in unit dosage form which comprises:

(a) blending as a % by weight on a dry weight basis:
(i) 23-28% by weight on a dry weight basis of DPP-IV inhibitor preferably LAF237, in free form or in acid addition salt form wherein at least 40%, preferably 60%, most preferably 80% even more preferably 90%, of the DPP-IV inhibitor especially vildagliptin has a particle size distribution of less than 250 μm or preferably between 10 to 250 μm;
(ii) 45-50% by weight on a dry weight basis of a pharmaceutically acceptable microcrystalline cellulose (Avicel PH 102);
(iii) 20-25% by weight on a dry weight basis of a pharmaceutically acceptable lactose;
(iv) 1.5-2% by weight on a dry weight basis of a pharmaceutically acceptable sodium starch glycolate; and
(v) 0.1-2% by weight on a dry weight basis of magnesium stearate,
   to form a DPP-IV inhibitor formulation in the form of a tableting powder, capable of being compressed preferably directly compressed into a tablet; and
   (b) compressing the formulation prepared during step (a) to form the compressed DPP-IV inhibitor tablet in unit dosage form.

Before the compression step (b) a sieving step is preferably applied to the formulation for basic delumping i.e. to get rid of any agglomerates/cakes.

In an other embodiment, the present invention covers capsule comprising a pharmaceutical composition such as the above described pharmaceutical compositions, and preferably wherein;
i) at least 60%, preferably 80% and most preferably 90% of the particles comprising the DPP-IV inhibitor preferably LAF237, in free form or in acid addition salt form, in the capsule have a particle size distribution between 10 to 500 μm,
ii) the water content of the tablet is less than 10% after 1 week at 25° C. and 60% RH.

More preferably capsule comprising a pharmaceutical composition such as the above described pharmaceutical compositions, and preferably wherein;
i) at least 40%, preferably 60%, most preferably 80% even more preferably 90% of the particles comprising the DPP-IV inhibitor preferably LAF237, in free form or in acid addition salt form, in the capsule have a particle size distribution of less than 250 μm preferably between 10 to 250 μm,
ii) the water content of the tablet is less than 5% after 1 week at 25° C. and 60% RH.

The final product is prepared in the form of tablets, capsules or the like by employing conventional tableting or similar machinery.

Most preferably the DPP-IV inhibitor for the herein described formulations, capsules, compressed tablets, uses or processes is selected from 1-{2-[(5-cyanopyridin-2-yl)amino]ethylamino}acetyl-2 (S)-cyano-pyrrolidine dihydrochloride, (S)-1-[(3-hydroxy-1-adamantyl)amino]acetyl-2-cyano-pyrrolidine, L-threo-isoleucyl thiazolidine, MK-0431, GSK23A, BMS-477118, 3-(aminomethyl)-2-isobuthyl-1-oxo-4-phenyl-1,2-dihydro-6-isoquinolinecarboxamide and 2-{[3-(aminomethyl)-2-isobuthyl-4-phenyl-1-oxo-1,2-dihydro-6-isoquinolyl]oxy}acetamide and optionally in any case pharmaceutical salts thereof.

Most preferably the DPP-IV inhibitor is 1-[3-hydroxy-adamant-1-ylamino)-acetyl]-pyrrolidine-2(S)-carbonitrile (LAF237 or vildagliptin) e.g. in amorphous state, or a crystalline form of vildagliptin.

Preferably the unit dosage form comprising vildagliptin e.g. tablet or capsule, contains between 10 and 150 mg of vildagliptin, preferably between 25 and 100 mg, most preferably between 50 and 100 mg of vildagliptin or its crystal form A. Preferably 50 mg or 100 mg of vildagliptin or its crystal form A.

Most preferably the herein described compositions), capsules, compressed tablets or direct compressed tablets, contain LAF237 in the form of its crystalline form preferably the crystal form “A” as defined hereinafter and preferably at least 1%, 5%, 10%, 20%, 30%, 40%, 50%, 60%, 70%, 80%, 90%, 95%, or 98% of the LAF237 compound can be in the form of a crystal form preferably the crystal form A.

The present invention also concerns a pharmaceutical composition (pharmaceutical formulation) capsules, compressed tablets or direct compressed tablets as described herein, comprising LAF237 in the form of its crystalline form preferably the crystal form “A” as defined hereinafter. In the formulation preferably at least 1%, 5%, 10%, 20%, 30%, 40%, 50%, 60%, 70%, 80%, 90%, 95%, or 98% of the LAF237 compound can be in the form of a crystal form preferably the crystal form “A”.

The present invention also concerns a composition (pharmaceutical formulation) e.g. as described herein, wherein less than 1% or less than 0.4% of LAF237 is in its “A” crystal form and more than 99% or 99.6% of LAF237 in its amorphous form.

Preferably at least 20 or 50%, most preferably at least 80% of the active ingredient LAF237 is in the form of its crystal form “A”.

Thus in a further aspect, present invention relates to the solid state physical properties of LAF237 (vildagliptin). These properties can be influenced by controlling the conditions under which LAF237 is obtained in solid form. Solid state physical properties include, for example, the flowability of the milled solid. Flowability affects the ease with which the material is handled during processing into a pharmaceutical product. When particles of the powdered compound do not flow past each other easily, a formulation specialist must take that fact into account in developing a tablet or capsule formulation, which may necessitate the use of glidants such as colloidal silicon dioxide, talc, starch or tribasic calcium phosphate.

Another important solid state property of a pharmaceutical compound is its rate of dissolution in aqueous fluid or on the bioavailability of the drug. The rate of dissolution of an active ingredient in a patient's stomach fluid can have therapeutic consequences since it imposes an upper limit on the rate at which an orally-administered active ingredient can reach the patient's bloodstream.

For example, different crystal forms or amorphous form of the same drug may have substantial differences in such pharmaceutically important properties as dissolution rates and bioavailability. Likewise, different crystals or amorphous form may have different processing properties, such as hydroscopicity, flowability, and the like, which could affect their suitability as active pharmaceuticals for commercial production.

The rate of dissolution is also a consideration in formulating syrups, elixirs and other liquid medicaments. The solid state form of a compound may also affect its behavior on compaction and its storage stability.

These practical physical characteristics are influenced by the conformation and orientation of molecules in the unit cell, which defines a particular polymorphic form of a substance. The polymorphic form may give rise to thermal behavior different from that of the amorphous material or another polymorphic form. Thermal behavior is measured in the laboratory by such techniques as capillary melting point, thermogravimetric analysis (TGA) and differential scanning calorimetry (DSC) and can be used to distinguish some polymorphic forms from others. A particular polymorphic form may also give rise to distinct spectroscopic properties that may be detectable by powder X-ray crystallography, solid state 13C NMR spectrometry and infrared spectrometry. Method used to characterize the crystal form: IR, X-ray powder diffraction, melting point determination.

During the development of the herein described formulations and particle size distribution, the applicant has discovered a novel crystal form of vildagliptin with unexpected good physicochemical characteristics which are particularly adapted to; improve the quality and preparation process of pharmaceutical formulations comprising vildagliptin (ease of processing, handling and dosing), improve the process to produce particles having an adapted particle size distribution especially for compressed tablets, improve the stability of vildagliptin in the formulations by improving e.g. the hygroscopic characteristics of vildagliptin, and to improve other properties such as bioavailability, solubility. These surprisingly good physicochemical characteristics render this new crystal form particularly suitable for the manufacture of various pharmaceutical dosage forms.

Thus in a first aspect, the present invention provides a process for preparing a crystalline form of vildagliptin or a salt thereof comprising the steps of:

i) heating a solution of vildagliptin or a salt thereof in an organic solvent,

ii) inducing the crystallization of vildagliptin, and

iii) recovering the crystalline vildagliptin.

In a preferred embodiment, the present invention provides a process for preparing the crystalline vildagliptin “Form A”, having an X-ray diffraction pattern, with peaks at 16.6°, 17.1°, 17.2°+/−0.3 degrees 2-theta or preferably at 12.0°, 13.5°, 16.6°, 17.1°, 17.2°, 20.1°, 22.5°, 27.4°, 28.1°+/−0.3 degrees 2-theta comprising the steps of:

i) heating a solution of vildagliptin in an organic solvent,

ii) inducing the crystallization of vildagliptin, and

iii) recovering the crystalline vildagliptin.

Preferably the solvent is selected from 2-butanone, 2-propanol/ethyl acetate, 2-propanol, acetone.

Preferably the crystallization comprises the step of;
i) heating a solution of LAF237 in an organic solvent, preferably selected from 2-butanone, 2-propanol/ethyl acetate, 2-propanol, acetone,
ii) cooling the solution to a temperature of about negative 20° C. to about 20° C., preferably to about negative 10° C. to about 10° C., to induce crystallization and
iii) recovering the crystalline vildagliptin preferably without heating.

Preferably as described above, after the heating step i) the temperature of the solution is reduced during the cooling step to a range of temperature of minus (−) 20° C. to about plus (+) 20° C., preferably to about minus (−) 10° C. to about (+) 10° C.

In an other embodiment the crystallization ii) can be induced by adding an anti-solvent to the solution (with or without cooling).

As used herein, an anti-solvent is a liquid that when added to a solution of compound X (i.e. vildagliptin) in the solvent, induces precipitation of X. Precipitation of X is induced by the anti-solvent when addition of the anti-solvent causes X to precipitate from the solution more rapidly or to a greater extent than X precipitates from a solution containing an equal concentration of X in the same solvent when the solution is maintained under the same conditions for the same period of time but without adding the anti-solvent. Precipitation can be perceived visually as a clouding of the solution or formation of distinct particles of X suspended in the solution or collected at the bottom the vessel containing the solution.

Preferably, the solution is cooled progressively to a temperature of about negative 20° C. to about 20° C., preferably to about negative 10° C. to about 10° C., to induce crystallization.

Preferably the solution is progressively cooled to about negative 20° C. to about 20° C., preferably to about negative 10° C. to about 10° C. to induce crystallization e.g cooled to 50° C. within a defined period of time, then to 30° C. within a defined period of time, then to 0° C. within a defined period of time.

Preferably the solution is progressively cooled to about negative 10° C. to about 10° C. within 100 to 500 minutes, preferably within 250 to 450 minutes.

Preferably the solution is cooled to 50° C. within 1 to 3 hours preferably 2 hours, then to 30° C. within 40 to 120 minutes preferably within 80 minutes, then to 0° C. within 30 to 120 minutes preferably 72 minutes.

The resulting crystals may then be recovered by techniques well known in the art, such as filtration, centrifugation, decanting, etc.

The crystals may then be dried. Drying may be carried out under ambient or reduced pressure. Preferably, drying is carried out at a temperature of from about 20° C. to about 60° C., more preferably in combination with a pressure of less than about 30 mm Hg.

Approximately a few hours of drying, e.g. about 2 to about 5 hours, depending on the conditions, may be sufficient.

As used herein, the term drying refers to removal of solvent through application of heat, preferably carried out under ambient or reduced pressure.

As used herein, the term reduced pressure refers to a pressure below one atmosphere, more preferably below about 100 mmHg.

As used herein, the term precipitation refers to formation of a suspension of small solid particles in a mixture.

As used herein, the term crystallization refers to a process for forming crystals from a liquid or gas.

The process as described above wherein at least 40% preferably 60% even preferably 80% of the resulting vildagliptin crystal “Form A” have a particle size distribution of less than 250 μm preferably of 10 to 250 μm.

In a further aspect, the present invention relates to a vildagliptin (LAF237) crystal “form A” which can be obtained by a process wherein;

i) a 1500 ml reactor, equipped with a mechanical stirrer, is charged with 120 grams (g) of LAF237 (1-[(3-Hydroxy-adamant-1-ylamino)-acetyl]-pyrrolidine-2(S)-carbonitrile), 3.6 g of Activated carbon, 2.4 g of cellflock 40, 3.6 g of 1,8-diazabicyclo[5.4.0]undec-7-ene and 483 g of 2-butanone.
ii) the mixture is heated to reflux (jacket temperature (JT): 95° C.) and stirred for 30 min,
iii) the mixture is filtered into a warm reactor (JT: 75° C.), the filter cake is washed with 48 g of 2-butanone;
iv) then IT is adjusted to 70° C. and a suspension of 0.102 g of the obtained re-crystallized LAF237 in 1.1 ml of 2-butanone is added to the solution,
v) the resulting suspension is stirred for 30 minutes (min.), cooled to internal temperature (IT) 50° C. within 2 h then to 30° C. within 80 min,
vi) finally the suspension is cooled to 0° C. within 72 min. and stirred for 1 additional hour,
vii) after this the suspension is filtered and the crude product is washed twice with a cold (0° C.) mixture of 37 g of 2-butanone and 34 g of t-butyl methyl ether,
viii) the crude product (vildagliptin crystal Form A) is finally dried under reduced pressure at about JT 55° C.

In a further aspect, the present invention concerns a crystalline form of vildagliptin.

The term “crystalline form of DPP-IV inhibitors” especially the term “a crystalline form of vildagliptin” according to the present invention also includes anhydrous crystalline form, partially crystalline form, mixture of several crystalline forms, hydrate crystalline forms or solvate crystalline forms.
Amorphous state: non-crystalline, (randomly ordered three-dimensional arrangement of atoms or molecules in solid state). The amorphous form of LAF237 was obtained by lyophilisation from water solution.
Crystalline state: Crystalline materials are three-dimensional periodic arrays of precise geometric arrangement of atoms or molecules.
Anhydrous crystalline forms: crystalline forms containing no solvent or water molecules in their three-dimensional periodic arrangement
Hydrates: crystalline forms containing one or more water molecules in their three-dimensional periodic arrangement
Solvates: crystalline forms containing one or more solvent molecules others than water in their three-dimensional periodic arrangement
Semi crystalline form: only partially ordered three-dimensional arrangement of atoms or molecules in solid state

In the present invention, the term a “with peaks” means “comprising peaks” and is not limitative.

In the present invention, the term a “polymorph or polymorphous” refers to a crystalline form which is different from the crystalline form “A”.

Preferably the present invention concerns a thermodynamically most stable crystalline form of vildagliptin (high physico-chemical stability).

Preferably the invention concerns a crystalline form of vildagliptin wherein 40% preferably 60% most preferably 80% of the vildagliptin crystal has a particle size distribution of less than 250 μM, preferably between 10 to 250 μm.

Preferably the herein described vildagliptin particles are crystalline vildagliptin “Form A” particles which preferably comprise more than 60% of crystalline vildagliptin “Form A”, most preferably more than 90% or 95% and even more preferably more than 98% of crystalline vildagliptin “Form A”.

By the term “thermodynamically most stable” the applicant means that different vildagliptin forms are investigated e.g. by solubility testing, heat of solution, DSC etc. against each other to detect the thermodynamical relationship (monotropy, enantiotropy) of the current crystalline form, between the different forms and which transitions occur. Based on this analysis it can be determined which is the most stable crystalline form e.g. at room temperature or over the whole temperature range.

In a preferred aspect, the present invention concerns a crystalline vildagliptin “Form A”, characterized by an X-ray diffraction pattern with peaks at about 16.6°, 17.1°, 17.2°+/−0.3 degrees 2-theta or preferably at about 12.0°, 13.5°, 16.6°, 17.1°, 17.2°, 20.1°, 22.5°, 27.4°, 28.1°+/−0.3 degrees 2-theta.

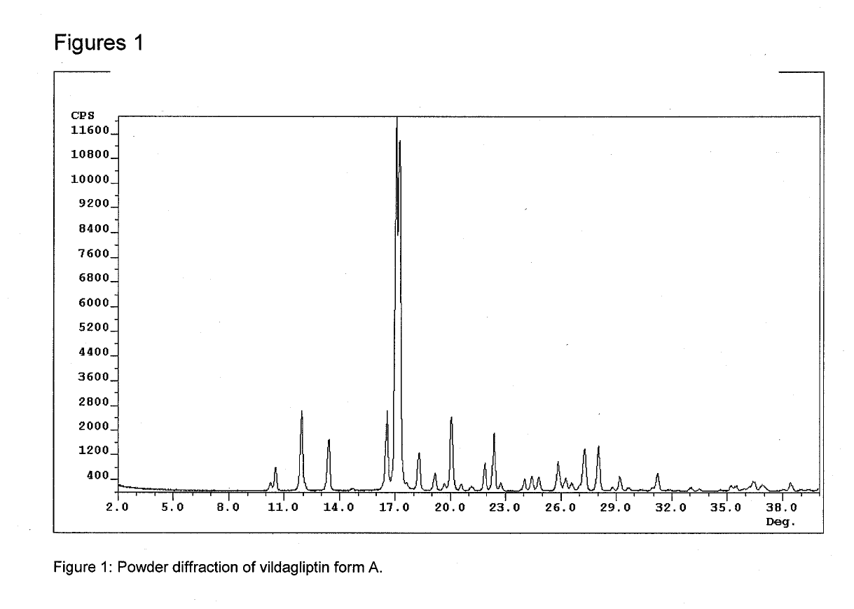

In a further aspects, the present invention concerns a crystalline vildagliptin “Form A” characterized by an X-ray powder pattern as substantially depicted in FIG. 1.

The X-ray data can be obtained by the method as described in the below example 7.2.i.

In a further aspects, the present invention concerns a crystalline vildagliptin “Form A” characterized by an IR spectrum in liquid paraffin having the following absorption significant bands expressed in reciprocal wave numbers (cm⁻¹) at; about 3293 cm⁻¹, 2925-2853 cm⁻¹, 2238 cm⁻¹, 1658 cm⁻¹, 1455/1354 cm⁻¹, 1254 cm⁻¹, 1121 cm⁻¹, 1054-1035 cm⁻¹, +/−2 cm⁻¹. FT-IR deviation: +/−2 cm⁻¹.

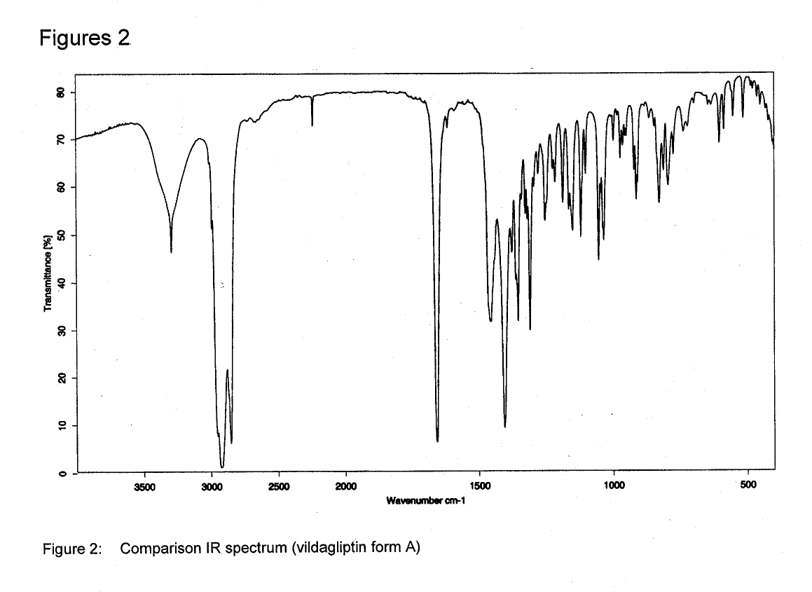

In a further aspects, the present invention concerns a crystalline vildagliptin “Form A” characterized by an IR spectrum in liquid paraffin having absorption bands expressed in reciprocal wave numbers (cm⁻¹) as substantially depicted in FIG. 2.

The IR (Infra Red) data can be obtained by the method described in example 7.2.ii.

In a further aspects, the present invention concerns a crystalline vildagliptin “Form A” characterized by melting point of 147° C.+/−4° C. (obtained e.g. by Differential Scanning Calorimetry (DSC) method, 10 K/min). Preferably around 149° C.+/−2° C.

In a further aspects, the present invention concerns a new LAF237 (vildagliptin) crystal form “Form A” characterized by a DSC thermogram that has no transitions between 25 C and 140 C while the amorphous form shows a glass transition at 27° C. (the sample change from dry to a paste) followed by a recrystallization exotherm starting at 50° C. and ending at 110° C. and subsequently a melt transition at about 127° C. Particularly wherein the melting point is lacking in the region of from about 140 C to about 150 C

In a preferred embodiment the vildagliptin crystal forms especially the crystal “Form A” have a particle size distribution of less than 250 μm preferably between 10 to 250 μm.

The present invention also concerns the use of a crystalline form of vildagliptin preferably the form A to produce the corresponding vildagliptin amorphous form or the use of a crystalline form of vildagliptin preferably the form A to produce another polymorphous form.

The present invention also concerns the use of the vildagliptin crystal form “Form A” to produce the corresponding vildagliptin amorphous form or the use of the vildagliptin crystal form “Form A” to produce another polymorphous form.

In a further aspect, the present invention relates to a process for the preparation of vildagliptin polymorphous forms wherein vildagliptin crystal form “Form A” is used as starting material or intermediate in the crystallization process.

The new crystalline Form A may be identified and differentiated by X-ray diffraction and/or infrared spectroscopy or any other method known in the art.

The vildagliptin crystal form “Form A” may be characterized by X-ray powder diffraction. The X-ray diffraction patterns are unique for the particular crystalline form. Each crystalline form exhibits a diffraction pattern with a unique set of diffraction peaks that can be expressed in 2 theta angles, d-spacing values and relative: peak intensities. 2 Theta diffraction angles and corresponding d-spacing values account for positions of various peaks in the X-ray powder diffraction pattern. D-spacing values are calculated with observed 2 theta angles and copper K(al) wavelength using the Bragg equation well known to those of skill in the art.

FIG. 1 shows an example of X-ray powder diffractogram of the crystalline Form A of vildagliptin. The X-ray data are obtained by the method as described in the below example 1.

The instrument measures the diffracted x-ray intensity (counts per second, cps) with respect to the angle of the x-ray source. Only crystalline samples diffract at well defined angles, thus sharp peaks are observed depending on the nature of the crystal form. Each form will give a unique diffraction pattern. The intensity of the peaks depend on particle size and shape, thus it is a property of the batch not of the crystalline form. The diffraction peaks (pattern) defines the location of each atom within the molecule and defines the crystal symmetry and space group for the given crystal system.

It should be kept in mind that slight variations in observed 2 theta angles or d-spacing values are expected based on the specific diffractometer employed, the analyst, and the sample preparation technique. More variation is expected for the relative peak intensities.

Identification of the exact crystal form of a compound should be based primarily on observed 2 theta angles with no importance attributed to relative peak intensities.

Since some margin of error is possible in the assignment of 2 theta angles and d-spacings, the preferred method of comparing X-ray powder diffraction patterns in order to identify a particular crystalline form is to overlay the X-ray powder diffraction pattern of the unknown form over the X-ray powder diffraction pattern of a known form.

For example, one skilled in the art can overlay an X-ray powder diffraction pattern of an unidentified crystalline form A of LAF237 obtained using the methods described herein, over FIG. 1 and readily determine whether the X-ray diffraction pattern of the unidentified form is substantially the same as the X-ray powder diffraction pattern of Form A. If the X-ray powder diffraction pattern is substantially the same as FIG. I, the previously unknown crystalline form of LAF237 can be readily and accurately identified as Form A. Although 2 theta angles or d-spacing values are the primary methods of identifying the crystalline form, it may be desirable to also compare relative peak 5 intensities. As noted above, relative peak intensities may vary depending upon the specific diffractometer employed and the analyst's sample preparation technique. The peak intensities are reported as intensities relative to the peak intensity of the strongest peak. The peak intensities is useful for quality control but should not be used for crystal form identification.

X-ray diffraction provides a convenient and practical means for quantitative determination of the relative amounts of crystalline and/or amorphous forms in a solid mixture. X-ray diffraction is adaptable to quantitative applications because the intensities of the diffraction peaks of a given compound in a mixture are proportional to the fraction of the corresponding powder in the mixture. The percent composition of crystalline LAF237 can be determined in an unknown composition.

Preferably, the measurements are made on solid powder LAF237. The X-ray powder diffraction patterns of an unknown composition can be compared to known quantitative standards containing pure crystalline forms of LAF237 Form A) to identify the percent ratio of the crystalline form A of LAF237. This is done by comparing the relative intensities of the peaks from the diffraction pattern of the unknown solid powder composition with a calibration curve derived from the X-ray diffraction patterns of pure known samples. The curve can be calibrated based on the X-ray powder diffraction pattern for the strongest peak from a pure sample of crystalline LAF237. The calibration curve may be created in a manner known to those of skill in the art. For example, five or more artificial mixtures of crystalline forms of LAF237, at different amounts, may be prepared. In a non-limiting example, such mixtures may contain, 2%, 5%, 7%, 8%, and 10% of LAF237 for each crystalline form. Then, X ray diffraction patterns are obtained for each artificial mixture using standard X-ray diffraction techniques. Slight variations in peak positions, if any, may be accounted for by adjusting the location of the peak to be measured. The intensities of the selected characteristic peak(s) for each of the artificial mixtures are then plotted against the known weight percentages of the crystalline form. The resulting plot is a calibration curve that allows determination of the amount of crystalline LAF237 in an unknown sample. For the unknown mixture of crystalline and amorphous LAF237, the intensities of the selected characteristic peak(s) in the mixture, relative to an intensity of this peak in a calibration mixture, may be used to determine the percentage of the given crystalline form in the composition, with the remainder determined to be the amorphous material.

The vildagliptin crystal form “Form A” may be also characterized by infrared spectroscopy. The infrared spectrum of crystalline Form A of vildagliptin obtained by the inventors is shown in FIG. 2. The IR (Infra Red) data of the present invention have been obtained by the method described in example 7.2.ii.

In a further aspect, the present invention concerns a pharmaceutical composition comprising vildagliptin crystal form “Form A”.

Preferably the formulation contains between 10 and 150 mg, preferably between 25 and 100 mg, most preferably between 50 and 100 mg of vildagliptin, preferably a crystal form of vildagliptin most preferably o the vildagliptin crystal form “Form A”, or a pharmaceutical salt thereof.

Preferably the present invention concerns a pharmaceutical composition or a compressed tablet as described herein above, comprising a vildagliptin crystal form preferably the vildagliptin crystal “Form A” or in any case a pharmaceutical salt thereof.

Preferably the vildagliptin crystal form or the vildagliptin crystal “Form A” is in the form of particles as herein described.

The pharmaceutical compositions comprising the vildagliptin crystal form “Form A”, according to the invention are those suitable for enteral, such as oral or rectal; transdermal and parenteral administration to mammals, including man, for the treatment of conditions mediated by DPP-4 inhibitors. Such conditions include those conditions mentioned hereinafter with respect to the treatment for which the compounds of the instant invention may be employed. The said pharmaceutical compositions comprise an effective amount of a pharmacologically active vildagliptin crystal “Form A” of the instant invention, alone or in combination with one or more pharmaceutically acceptable carriers.

The pharmacologically vildagliptin crystal “Form A” of the invention may be employed in the manufacture of pharmaceutical compositions comprising an effective amount thereof in conjunction or admixture with excipients or carriers suitable for either enteral or parenteral application. Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, preferably about 1-50%, of the vildagliptin crystal “Form A”.

Suitable formulations for transdermal application include a therapeutically effective amount of a compound of the invention with carrier. Advantageous carriers include absorbable pharmacologically acceptable solvents to assist passage through the skin of the host. Characteristically, transdermal devices are in the form of a bandage comprising a backing member, a reservoir containing the compound optionally with carriers, optionally a rate controlling barrier to deliver the compound of the skin of the host at a controlled and predetermined rate over a prolonged period of time, and means to secure the device to the skin.

The vildagliptin crystal “Form A” or the pharmaceutical compositions comprising the vildagliptin crystal form “Form A” as defined above, can be administered either alone or in a combination with another (e.g. one or two) therapeutic agent (in the same or in different dosage unit), e.g., each at an effective therapeutic dose as reported in the art. The herein described compressed tablets or directly compressed tablets or formulations can as well comprise a further therapeutic agent. Such therapeutic agents include insulin, insulin derivatives and mimetics; insulin secretagogues such as the sulfonylureas, e.g., Glipizide and Amaryl; insulinotropic sulfonylurea receptor ligands, such as meglitinides, e.g., nateglinide and repaglinide; insulin sensitizers, such as protein tyrosine phosphatase-1B (PTP-1B) inhibitors, GSK3 (glycogen synthase kinase-3) inhibitors or RXR ligands; biguanides, such as metformin; glitazones such as pioglitazone or rosiglitazone, alpha-glucosidase inhibitors, such as acarbose; GLP-1 (glucagon like peptide-1), GLP-1 analogs, such as Exendin-4, and GLP-1 mimetics; DPPIV (dipeptidyl peptidase IV) inhibitors, e.g. isoleucin-thiazolidide; DPP728 and LAF237, hypolipidemic agents, such as 3-hydroxy-3-methyl-glutaryl coenzyme A (HMG-CoA) reductase inhibitors, e.g., lovastatin, pitavastatin, simvastatin, pravastatin, cerivastatin, mevastatin, velostatin, fluvastatin, dalvastatin, atorvastatin, rosuvastatin, fluindostatin and rivastatin, squalene synthase inhibitors or FXR (liver X receptor) and LXR (farnesoid X receptor) ligands, cholestyramine, fibrates, nicotinic acid, valsartan and aspirin. A LAF237 crystal form A of the present invention may be administered either simultaneously, before or after the other active ingredient, either separately by the same or different route of administration or together in the same pharmaceutical formulation (same dosage unit).

The vildagliptin crystal “Form A” is preferably administered in combination with one or two compounds selected from metformin, a glitazone (such as pioglitazone or rosiglitazone), insulin, sulfonylureas, nateglinide, or valsartan.

In a further aspect, the present invention concerns the use of the herein described formulations, capsules, tablets, compressed tables, direct compressed tablets for the treatment of conditions, such as non-insulin-dependent diabetes mellitus, arthritis, obesity, allograft transplantation, calcitonin-osteoporosis, Heart Failure, Impaired Glucose Metabolism), IGT (Impaired Glucose Tolerance), neurodegenerative diseases such as Alzheimer's and Parkinson disease, modulating hyperlipidemia, modulating conditions associated with hyperlipidemia or for lowering VLDL, LDL and Lp(a) levels, cardiovascular or renal diseases e.g. diabetic cardiomyopathy, left or right ventricular hypertrophy, hypertrophic medial thickening in arteries and/or in large vessels, mesenteric vasculature hypertrophy, mesanglial hypertrophy, neurodegenerative disorders and cognitive disorders, to produce a sedative or anxiolytic effect, to attenuate post-surgical catabolic changes and hormonal responses to stress, to reduce mortality and morbidity after myocardial infarction, the treatment of conditions related to the above effects which may be mediated by GLP-1 and/or GLP-2 levels.

In a further aspect, the present invention concerns an immediate release dosage form, wherein the average DPP-4 inhibition, 10.5 hours after a once daily administration of 50 mg of vildagliptin or a salt thereof, is at least 79% preferably at least 83% or between 83% and 94.5%, or 89.34+/−3.02%.

An immediate release dosage form, wherein the average DPP-4 inhibition, between 0.25 and 10.5 hours after a once daily administration of 50 mg of vildagliptin or a salt thereof, is between 84% and 98%.

An immediate release dosage form, wherein the average DPP-4 inhibition over 24 hours after a once daily administration of 50 mg of vildagliptin or a salt thereof, is of 64.2%+/−12.7%.

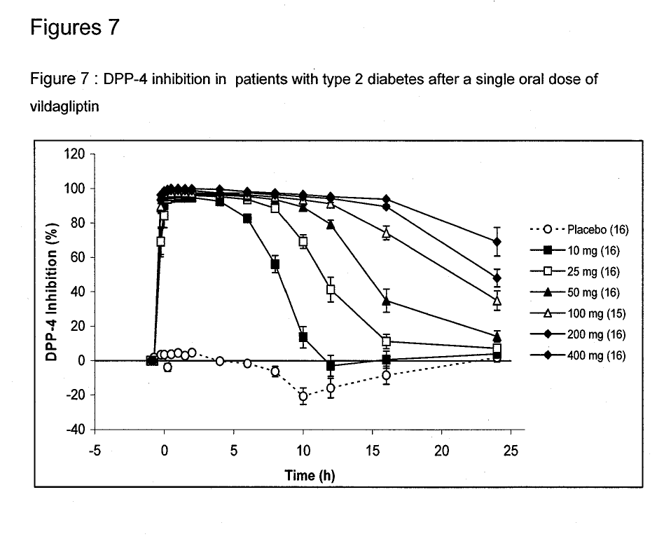

An immediate release dosage form, wherein the DPP-4 inhibition over 24 hours after a once daily administration of 50 mg of vildagliptin or a salt thereof, is as substantially depicted in FIG. 7.

An immediate release dosage form as described above, wherein the dosage form is any of the herein described formulations, tablets or capsules.

The present invention also concerns an immediate release dosage form, wherein the average DPP-4 inhibition, 10.5 hours after a once daily administration of 100 mg of vildagliptin or a salt thereof, is at least 83% preferably at least 90% or between 90% and 95.2%.

An immediate release dosage form, wherein the average DPP-4 inhibition, between 0.25 and 10.5 hours after a once daily administration of 100 mg of vildagliptin or a salt thereof, is between 84% and 98.8%.

An immediate release dosage form, wherein the average DPP-4 inhibition over 24 hours after a once daily administration of 100 mg of vildagliptin or a salt thereof, is of 76.3%+/−13.7%.

An immediate release dosage form, wherein the DPP-4 inhibition over 24 hours after a once daily administration of 100 mg of vildagliptin or a salt thereof, is as substantially depicted in FIG. 7.

An immediate release dosage form as described above, wherein the dosage form is any of the herein described and claimed pharmaceutical compositions, tablets, compressed tablets.

An immediate release dosage form as described above, wherein the dosage form is administered to a patient with type 2 diabetes.

An immediate release dosage form, wherein the average DPP-4 inhibition over 10 hours after a twice daily administration of 50 mg of vildagliptin or a salt thereof, is at least 75% preferably 80%.

An immediate release dosage form, wherein the average DPP-4 inhibition over 24 hours after a twice daily administration of 50 mg of vildagliptin or a salt thereof, is at least 50% preferably 60% or 64.2%.

An immediate release formulation, wherein the average DPP-4 inhibition over 10 hours after a twice daily administration of 50 mg of vildagliptin or a salt thereof, is at least 70% preferably 80%.

An immediate release formulation, wherein the average DPP-4 inhibition over 24 hours after a twice daily administration of 50 mg of vildagliptin or a salt thereof, is at least 60% preferably 70% or 76.3%.

An immediate release dosage form as described above, wherein the dosage form is any of the herein described and claimed pharmaceutical compositions, tablets, compressed tablets.

The term “a twice daily administration of 50 mg of vildagliptin or a salt thereof” means two separate administration vildagliptin, wherein the second administration is taken between 8 to 12 hours after the first administration, preferably between 9 and 11 hours after the first 50 mg administration.

The term “an immediate release dosage form” means a dosage form wherein the arithmetic mean tₘₐₓ of vildagliptin is of 2.0 hr+/−1.9 hr or +/−1.4 hr following oral administration of a single dose of 25 to 200 mg of vildagliptin.

In another embodiment, the present invention provides;

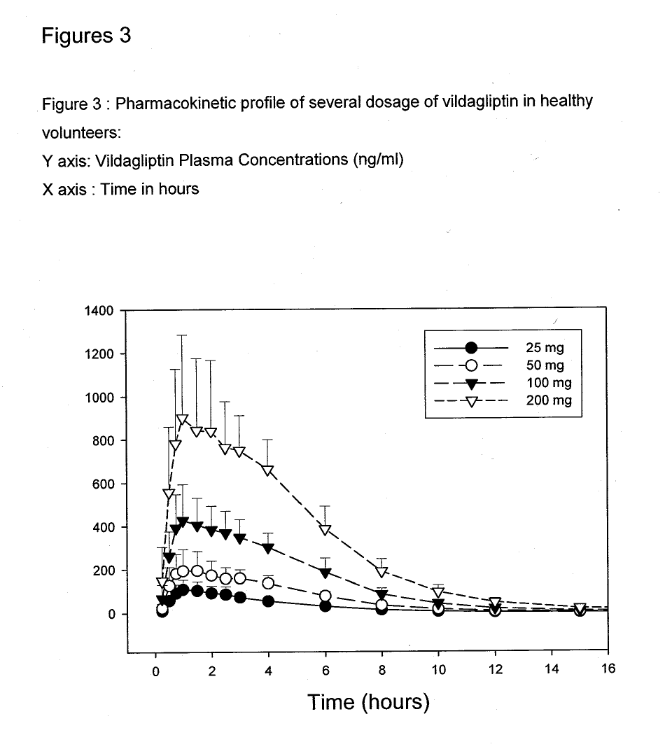

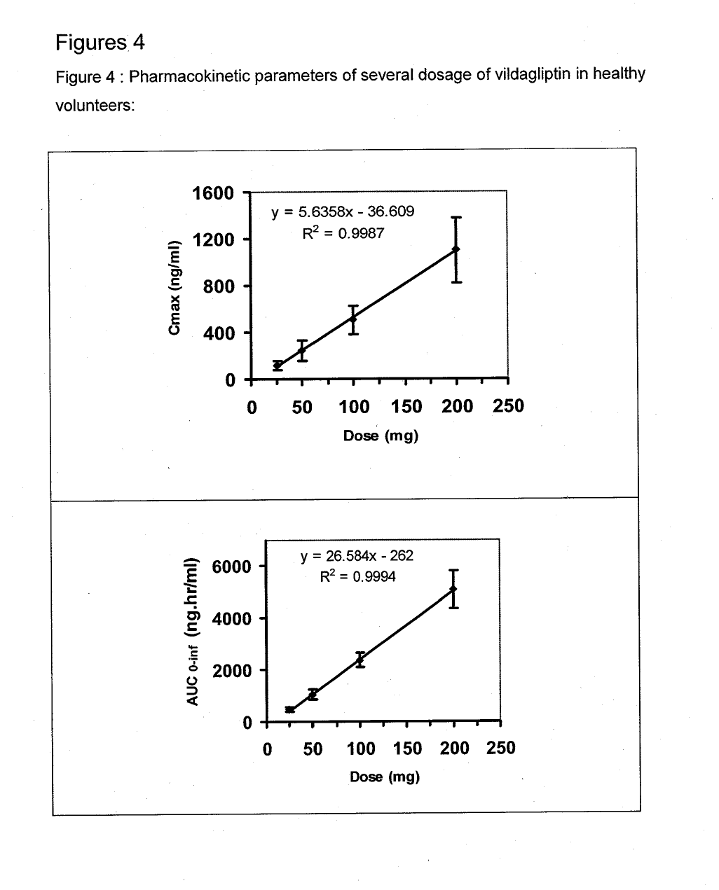

i) a solid oral dosage form comprising about 50 mg of vildagliptin free base, or a respective amount of a pharmaceutically acceptable salt thereof, and a carrier medium, said dosage form providing an arithmetic mean maximum plasma concentration of vildagliptin ranging from about 77.3 ng/mL+/−20.8 ng/mL to about 195 ng/mL+/−89.1 ng/mL between about 0.5 and about 6 hours following oral administration of a single 50 mg dose of vildagliptin.
ii) a solid oral dosage form comprising about 50 mg vildagliptin free base, or a respective amount of a pharmaceutically acceptable salt thereof, and a carrier medium, said dosage form providing an arithmetic mean AUC_{(0-∞)} of vildagliptin ranging from about 839 to about 1221 ng·h/mL i.e. 1030 ng·h/mL+/−191 ng·h/mL following oral administration of a single dose of 50 mg of vildagliptin.
iii) a solid oral dosage form comprising about 50 mg vildagliptin free base, or a respective amount of a pharmaceutically acceptable salt thereof, and a carrier medium, said dosage form providing an arithmetic mean tₘₐₓ of vildagliptin of 2.1 hr+/−1.3 hr following oral administration of a single dose of 50 mg of vildagliptin.
iv) a solid oral dosage form comprising about 50 mg vildagliptin free base, or a respective amount of a pharmaceutically acceptable salt thereof, and a carrier medium, wherein said dosage form provides:
an arithmetic mean maximum plasma concentration of vildagliptin ranging from about 77.3 ng/mL+/−20.8 ng/mL to about 195 ng/mL+/−89.1 ng/mL between about 0.5 and about 6 hours following oral administration of a single 50 mg dose of vildagliptin, and/or
an arithmetic mean AUC_{(0-∞)} of vildagliptin ranging from about 839 to about 1221 ng·h/mL i.e. 1030 ng·h/mL+/−191 ng·h/mL following oral administration of a single dose of 50 mg of vildagliptin, and/or
an arithmetic mean tₘₐₓ of vildagliptin of 2.1 hr+/−1.3 hr following oral administration of a single dose of 50 mg of vildagliptin.
   v) a solid oral dosage form comprising about 50 mg vildagliptin free base, or a respective amount of a pharmaceutically acceptable salt thereof, and a carrier medium, said dosage form providing a pharmacokinetic profile as substantially depicted in FIG. 3 or 4, following oral administration of a single dose of 50 mg of vildagliptin.

Preferably the administration of the oral dosage is performed in a healthy human subject.

In another embodiment, the present invention provides;

i) a solid oral dosage form comprising about 100 mg of vildagliptin free base, or a respective amount of a pharmaceutically acceptable salt thereof, and a carrier medium, said dosage form providing an arithmetic mean maximum plasma concentration of vildagliptin ranging from about 186 ng/mL+/−64.9 ng/mL to about 428 ng/mL+/−165 ng/mL between about 0.5 and about 6 hours following oral administration of a single 50 mg dose of vildagliptin.
ii) a solid oral dosage form comprising about 100 mg vildagliptin free base, or a respective amount of a pharmaceutically acceptable salt thereof, and a carrier medium, said dosage form providing an arithmetic mean AUC_{(0-∞)} of vildagliptin ranging from about 2071 to about 2629 ng·h/mL i.e. 2350 ng·h/mL+/−279 ng·h/mL following oral administration of a single dose of 100 mg of vildagliptin.
iii) a solid oral dosage form comprising about 100 mg vildagliptin free base, or a respective amount of a pharmaceutically acceptable salt thereof, and a carrier medium, said dosage form providing an arithmetic mean tₘₐₓ of vildagliptin of 2.0 hr+/−1.4 hr following oral administration of a single dose of 100 mg of vildagliptin.
iv) a solid oral dosage form comprising about 100 mg vildagliptin free base, or a respective amount of a pharmaceutically acceptable salt thereof, and a carrier medium, wherein said dosage form provides:
an arithmetic mean maximum plasma concentration of vildagliptin ranging from about 186 ng/mL+/−64.9 ng/mL to about 428 ng/mL+/−165 ng/mL between about 0.5 and about 6 hours following oral administration of a single 50 mg dose of vildagliptin, and/or
an arithmetic mean AUC_{(0-∞)} of vildagliptin ranging from about 2071 to about 2629 ng·h/mL i.e. 2350 ng·h/mL+/−279 ng·h/mL following oral administration of a single dose of 100 mg of vildagliptin, and/or
an arithmetic mean tₘₐₓ of vildagliptin of 2.0 hr+/−1.4 hr following oral administration of a single dose of 100 mg of vildagliptin.
   v) a solid oral dosage form comprising about 100 mg vildagliptin free base, or a respective amount of a pharmaceutically acceptable salt thereof, and a carrier medium, said dosage form providing a pharmacokinetic profile as substantially depicted in FIG. 3 or 4, following oral administration of a single dose of 100 mg of vildagliptin.

Preferably the administration of the oral dosage is performed in a healthy human subject.

In another embodiment, the present invention provides;

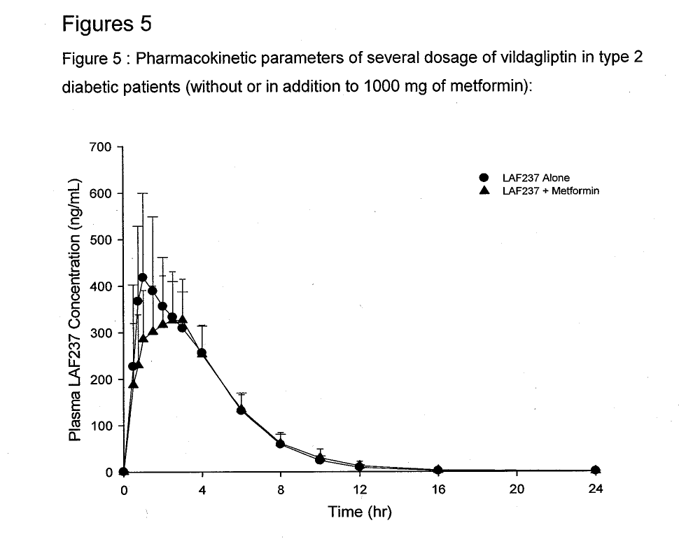

i) a solid oral dosage form comprising about 100 mg of vildagliptin free base, or a respective amount of a pharmaceutically acceptable salt thereof, and a carrier medium, said dosage form providing an arithmetic mean maximum plasma concentration of vildagliptin ranging from about 188 ng/mL+/−132 ng/mL to about 327 ng/mL+/−87.6 ng/mL between about 0.5 and about 6 hours following oral administration of a single 100 mg dose of vildagliptin, concomitantly with 1000 mg of metformin.
ii) a solid oral dosage form comprising about 100 mg vildagliptin free base, or a respective amount of a pharmaceutically acceptable salt thereof, and a carrier medium, said dosage form providing an arithmetic mean AUC_{(0-24 h)} of vildagliptin of 1840 ng·h/mL+/−360 ng·h/mL following oral administration of a single dose of 100 mg of vildagliptin, concomitantly with 1000 mg of metformin.
iii) a solid oral dosage form comprising about 100 mg vildagliptin free base, or a respective amount of a pharmaceutically acceptable salt thereof, and a carrier medium, said dosage form providing an arithmetic mean tₘₐₓ of vildagliptin of 2.5 hr+/−1.3 hr following oral administration of a single dose of 100 mg of vildagliptin, concomitantly with 1000 mg of metformin.
iv) a solid oral dosage form comprising about 100 mg vildagliptin free base, or a respective amount of a pharmaceutically acceptable salt thereof, and a carrier medium, wherein said dosage form provides:
an arithmetic mean maximum plasma concentration of vildagliptin ranging from about 188 ng/mL+/−132 ng/mL to about 327 ng/mL+/−87.6 ng/mL between about 0.5 and about 6 hours following oral administration of a single 100 mg dose of vildagliptin, concomitantly with 1000 mg of metformin, and/or
   an arithmetic mean AUC_{(0-24 h)} of vildagliptin of 1840 ng·h/mL+/−360 ng·h/mL following oral administration of a single dose of 100 mg of vildagliptin, concomitantly with 1000 mg of metformin, and/or
   an arithmetic mean tₘₐₓ of vildagliptin of 2.5 hr+/−1.3 hr following oral administration of a single dose of 100 mg of vildagliptin, concomitantly with 1000 mg of metformin.
      v) a solid oral dosage form comprising about 100 mg vildagliptin free base, or a respective amount of a pharmaceutically acceptable salt thereof, and a carrier medium, said dosage form providing a pharmacokinetic profile as substantially depicted in FIG. 5, following oral administration of a single dose of 100 mg of vildagliptin, concomitantly with 1000 mg of metformin.

Preferably the administration of the oral dosage is performed in a human subject with type 2 diabetes.

In another embodiment, the present invention provides;

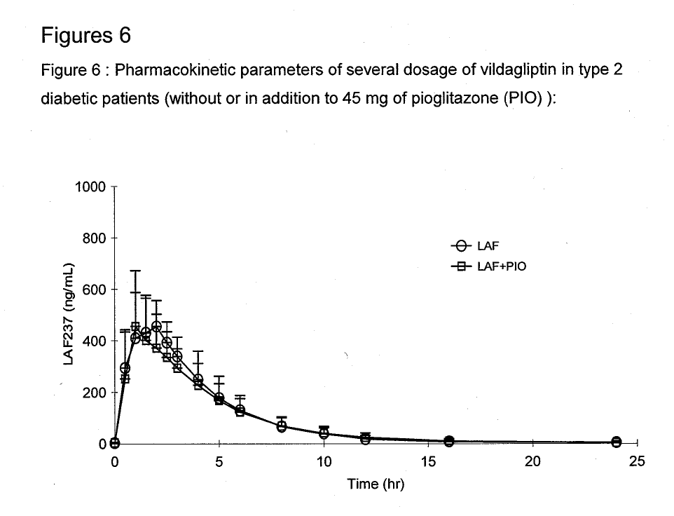

i) a solid oral dosage form comprising about 100 mg of vildagliptin free base, or a respective amount of a pharmaceutically acceptable salt thereof, and a carrier medium, said dosage form providing an arithmetic mean maximum plasma concentration of vildagliptin ranging from about 123 ng/mL+/−51.5 ng/mL to about 455 ng/mL+/−217 ng/mL between about 0.5 and about 6 hours following oral administration of a single 100 mg dose of vildagliptin, concomitantly with 45 mg of pioglitazone.
ii) a solid oral dosage form comprising about 100 mg vildagliptin free base, or a respective amount of a pharmaceutically acceptable salt thereof, and a carrier medium, said dosage form providing an arithmetic mean AUC_{(0-∞)} of vildagliptin of 2090 ng·h/mL+/−446 ng·h/mL following oral administration of a single dose of 100 mg of vildagliptin, concomitantly with 45 mg of pioglitazone.
iii) a solid oral dosage form comprising about 100 mg vildagliptin free base, or a respective amount of a pharmaceutically acceptable salt thereof, and a carrier medium, said dosage form providing an arithmetic mean tₘₐₓ of vildagliptin of 1 hr+/−1.3 hr following oral administration of a single dose of 100 mg of vildagliptin, concomitantly with 45 mg of pioglitazone.
iv) a solid oral dosage form comprising about 100 mg vildagliptin free base, or a respective amount of a pharmaceutically acceptable salt thereof, and a carrier medium, wherein said dosage form provides:
an arithmetic mean maximum plasma concentration of vildagliptin ranging from about 123 ng/mL+/−51.5 ng/mL to about 455 ng/mL+/−217 ng/mL between about 0.5 and about 6 hours following oral administration of a single 100 mg dose of vildagliptin, concomitantly with 45 mg of pioglitazone, and/or,
   an arithmetic mean AUC_{(0-∞)} of vildagliptin of 2090 ng·h/mL+/−446 ng·h/mL following oral administration of a single dose of 100 mg of vildagliptin, concomitantly with 45 mg of pioglitazone, and/or
   an arithmetic mean tₘₐₓ of vildagliptin of 1 hr+/−1.3 hr following oral administration of a single dose of 100 mg of vildagliptin, concomitantly with 45 mg of pioglitazone.
      v) a solid oral dosage form comprising about 100 mg vildagliptin free base, or a respective amount of a pharmaceutically acceptable salt thereof, and a carrier medium, said dosage form providing a pharmacokinetic profile as substantially depicted in FIG. 6, following oral administration of a single dose of 100 mg of vildagliptin, concomitantly with 45 mg of pioglitazone.

Preferably the administration of the oral dosage is performed in a human subject with type 2 diabetes.

A solid oral dosage form comprising about 100 mg or 50 mg of vildagliptin as described in the above sections i) to v), wherein the dosage form is in the form of one of the herein described and claimed pharmaceutical compositions, tablets, compressed tablets.

In each case in particular in the compound claims, the final products of the working examples, the subject matter of the final products, the analytical and measurement methods (e.g. USP documents) the methods to obtain the right particles size, the pharmaceutical preparations, the excipients and the claims are hereby incorporated into the present application by reference to the herein mentioned publications or patent applications.

The invention is illustrated in particular by the examples and also relates to the new compounds named in the examples and to their usage and to methods for the preparation thereof.

The following examples serve to illustrate the invention without limiting the invention in any way.

### EXAMPLE 1

To prepare the 25 mg tablet size (directly compressed tablet), a batch size of 7 kg is prepared using amounts corresponding to the following per unit: 25 mg per unit of the compound 1-[3-hydroxy-adamant-1-ylamino)acetyl]-pyrrolidine-2(S)-carbonitrile is mixed with 35.1 mg of microcrystalline cellulose, 17.5 mg anhydrous lactose and 1.6 mg sodium starch glycolate. The ingredients are pre-blended together in a commercial bin blender, then sieved through a 500 μm or 850 μm screen. The mix is blended again in the bin blender, then the necessary amount of the magnesium stearate to yield the 0.8 mg magnesium stearate per 25 mg tablet size, is added. Blending in each step is conducted at about 150-45° rotations, to ensure homogeneity of the mixture. Following blending again in the bin blender, the mix can be tabletted in a conventional tableting machine. The individual tablet weight for the 25 mg tablet is 80 mg. Tablets having 50 mg active ingredient weigh 160 mg, and 100 mg active ingredient tablets weigh 320 mg, respectively. The blend is a powder which has excellent compressibility into the desired tablet size.

### EXAMPLE 2

The same process as described above in example 1, can be applied to produce the below described preferred 50 mg tablet (directly compressed).

| | Composition per | Quantity per |
|---|---|---|
| Components | unit (mg) | batch (kg) |
| LAF 237 drug substance | 50.00 | 65.0 |
| Microcrystalline cellulose, PH102 | 95.68 | 124.38 |
| (Ph. Eur., NF) | | |
| Lactose anhydrous DT (USP, Ph. Eur.) | 47.82 | 62.17 |
| Sodium starch glycolate (USP, Ph. Eur.) | 4.00 | 5.2 |
| Magnesium stearate (Ph. Eur, NF) | 2.50 | 3.25 |
| Total weight, per tablet or per batch | 200.0 | 260.0 |

Equivalent 100 mg tablets of LAF237 are produced i.e. 100 mg of LAF237, 191,36 mg of Microcrystalline cellulose, 95.64 mg of Lactose anhydrous, 8 mg of Sodium starch glycolate, 5 mg of Magnesium stearate.

### EXAMPLE 3

The tablets prepared in accordance with the above Description and examples can be tested as follows.

### Tablet Evaluation Methods

1. Average tablet weight. Twenty tablets are weighed on an analytical balance and the average tablet weight calculated.
2. Tablet breaking strength (kilo bond-kp). 5 tablets are individually tested using a Schleuniger crushing strength tester, and the average breaking strength calculated.
3. Friability (% loss). 10 tablets, accurately weighed, are subjected to 10 minutes friability testing using a Roche Friabilator. The tablets are dedusted, reweighed, and the weight loss due to the friability is calculated as a percentage of the initial weight.
4. Dispersion Disintegration time DT (The test for dispersible tablets defined in the British Pharmacopoeia, 1988, Volume II, page 895—BP 1988). 6 tablets are tested in accordance to the above-defined BP test (without discs) for dispersible tablets. This utilizes water at a temperature of 19°-21° C.
5. Dispersion Quality. In accordance with the BP uniformity of dispersion test for dispersible tablets (BP 1988 Volume II page 895), two tablets are placed in 100 ml of water at 19°-21° C. and allowed to disperse.

### Granule Evaluation Methods

1. Loss on Drying (LOD). The residual moisture content of the granule (LOD) can be determined on a 3-4 g sample using a Computrac moisture analyser set at 90° C. operated in accordance with the manufacturer's procedure.
2. Weight Median Diameter (WMD). A 10 g sample of granule is sifted for 2 minutes at suitable pulse and sift amplitudes in an Allen Bradley sonic sifter in accordance with manufacturer's instructions. Sieves of 300 μm, 250 μm, 200 μm, 150 μm, 100 μm, 53 μm and 40 μm are used. The WMD is calculated from the cumulative percentage undersize size distribution using a computer program.

### EXAMPLE 4

### Improved Manufacturing Robustness

A preliminary compactibility assessment is carried out on a Carver press using different formulations as well as binary mixtures of LAF 237 with different excipients e.g. microcrystalline cellulose (Avicel PH102).

Data demonstrate that our claimed compositions on being compressed with increasing levels of pressure (compression force) show a substantially useful increase in tablet strength. In particular e.g. mixture of LAF237 and Avicel show a substantially useful increase in tablet strength. These results indicated that from compactibility point of view microcrystalline cellulose e.g. Avicel would a preferred excipient to be combined with LAF237. With increasing pressure (compression force) our claimed formulations and selected ranges show a substantially useful increase in tablet strength.

A compactibility study is carried out on an instrumented Korsch single station press with force and displacement sensors on both upper and lower punches.

A clear indication is afforded from these data that LAF237 tablets are very likely to have poor tablet hardness/crushing strength unless diluted out using sufficient filler with excellent compactibility. Our claimed formulations and selected ranges are particularly adapted to provide the required compactibility. Microcrystalline cellulose e.g. Avicel is a good choice for a filler in this respect.

### EXAMPLE 5

### Friability

Evaluation is carried out using a Manesty Betapress at 6 different settings: strain rate settings of 66-90 rpm (63,000-86,000 TPH) and force of 7.5-15 kN. The trials uses Flat-faced Beveled-edge (FFBE) tooling of 9 mm diameter for 250 mg tablets and 10 mm diameter for 310 mg tablets (other diameters are used depending on the weight of the tested tablet). Total tablet weights were selected so that both the 9 and 10 mm FFBE tablets would have 100 mg of LAF237 and identical tablet thickness. Friability, Compression profile, Strain rate profile and Weight variation are the measured outcomes. Study design and the friability results obtained from the study are used to determine the variables (particle size distribution in the formulation, tablet weight, tablet thickness and weight, water content in the tablet etc) impacting the outcome of hardness.

### EXAMPLE 7

### Particle Size Distribution and Crystal Form A (Non Limitative Example)

The vildagliptin particle size distribution between 10 to 250 μm, which is particularly adapted to produce the herein described formulations especially the compressed tablets, can be produced as described below

### 1. Preparation of Particle Size Distribution Via a Crystal Form of Vildagliptin Applied for Direct Compression Tablets

The applicant has discovered a particle size distribution (between 10 to 250 μm) of vildagliptin, which is particularly suitable for direct compression tablets. The particle size distribution determined by laser light diffraction or equivalent method is specified as follows: ×10 larger or equal 5 μm, ×50 larger or equal 35 μM and ×90 less or equal 380 μm.

Particle size have been measured by Fraunhofer light diffraction.
Reagents used:

Dispersing aid: e.g. Antistatic Additive AA3, Shell, approx. 1% in hexane.

Dispersion liquid: e.g. iso-hexane, Merck cat. no. 1.04333 with approx. 1 ml dispersing aid.

### Equipment:

Measuring device: e.g. Sympatec HELOS, Sympatec GmbH, Germany

Dispersion device: Suspension cell, e.g. QUIXEL, Sympatec GmbH, Germany

### Conditions

Focal length: 1000 mm; Optical concentration: ≧5%; Duration of measurement: 60 s; Flow through cuvette: 6 mm; Pump speed: 15-30%; Ultrasonication time: 30 s.

### Procedure:

Stock dispersion: To about 0.5 g of test substance add some drops of the dispersing aid. Mix intensively, e.g. on a vortex mixer, in order to wet the substance thoroughly and to form a smooth and homogeneous paste. Dilute the paste with dispersion liquid to a final volume of 3-6 ml and mix the dispersion again.

Measurement: Prepare the test dispersion and determine the cumulative volume distribution using a laser light diffraction instrument in accordance with the instruction manual. The parameters may be adjusted so that the test dispersion is representative, homogeneous and well dispersed.

Evaluation/assessment: Determine the particle sizes at the undersize values of 10%, 50% and 90% (x₁₀, x₅₀, x₉₀), and additional values in question, from the cumulative volume distribution.

This particle size distribution can be obtained by the below described process. This particle size distribution can be obtained with any form of vildagliptin such as from amorphous vildagliptin, or a crystalline forms of vildagliptin, preferably the vildagliptin crystal form A.

The below non limitative example combines the preparation of the vildagliptin crystal form A and a subsequent mechanical stress.

### A. Preparation of the Vildagliptin Crystal Form A

A 1500 ml reactor, equipped with a mechanical stirrer, is charged with 120 grams (g) of vildagliptin (1-[(3-Hydroxy-adamant-1-ylamino)-acetyl]-pyrrolidine-2(S)-carbonitrile), 3.6 g of Activated carbon, 2.4 g of cellflock 40, 3.6 g of 1,8-diazabicyclo[5.4.0]undec-7-ene and 483 g of 2-butanone.

The mixture is heated to reflux (jacket temperature (JT): 95° C.) and stirred for 30 min. The mixture is filtered into a warm (JT: 75° C.) reactor, the filter cake is washed with 48 g of 2-butanone.

Then IT is adjusted to 70° C. and a suspension of 0.102 g of the obtained re-crystallized vildagliptin in 1.1 ml of 2-butanone is added to the solution. The resulting suspension is stirred for 30 minutes (min.), cooled to internal temperature (IT) 50° C. within 2 h then to 30° C. within 80 min. Finally the suspension is cooled to 0° C. within 72 min. and stirred for 1 additional hour. After this the suspension is filtered and the crude product is washed twice with a cold (0° C.) mixture of 37 g of 2-butanone and 34 g of t-butyl methyl ether.

The crude product (crystal Form A) is finally dried under reduced pressure at about JT 55° C.

The resulting particle size distribution of the vildagliptin crystal form “A” has physical characteristics which are particularly adapted to be able to obtain the desired particle size distribution by the subsequent milling step. The obtained substance is a white to off white crystalline powder.

### B. Mechanical Stress

The material in the desired particle size range can be produced from amorphous vildagliptin, crystalline forms of vildagliptin, crystalline form of vildagliptin, crystalline form “A” of vildagliptin, a partially crystalline form of vildagliptin, a polymorphous form of vildagliptin, a solvate form of vildagliptin, or an hydrate form of vildagliptin, by mechanical stress. This stress can be mediated by impact, shear or compression. Preferably the crystalline form “A” of vildagliptin has been used.

In most commercially available grinding equipment a combination of these principles occurs. For the LAF237 crystals obtained by the above described crystallization process preferably a mechanical impact or jet mill is used. The most preferable mechanical impact mill can be equipped with different kind of beaters, screens, liners or with pin plates. For our process preferably an impact mill with plate beater and a slit screen 5*2.5 cm is used. The impact speed should be variable between 20 and 100 m/s (as peripheral speed) to adapt to any batch to batch variation. In our case a peripheral speed of the beater of about 40-50 m/s is used.

Best results (particle size distribution) where obtained by combining the preparation a crystal form of vildagliptin preferably the form “A” and a subsequent mechanical stress e.g. roller compaction, milling and/or sieving.

### 2. Characterization of the Crystal Form A.

### i) X-Ray Powder Diffraction (XRPD)

The powder diffractometer used is the Type XDS 2000 or X1, Scintag, Santa Clara, USA.

Procedure: The test substance is placed on the specimen holder. The X-ray diffraction pattern is recorded between 2° and 35° (2 theta) with Cu K_{α} radiation (45 kV, 40 mA). The measurements are performed at about 45 kV and 40 mA under the following conditions:
Scan rate: 0.5° (2 theta)/min
Chopper increment: 0.02°
Slits (from left to right): 2, 3, 0.3, 0.2 mm

The positions of all the lines in the X-ray diffraction pattern of the test substance with those in the X-ray diffraction pattern of the reference substance are compared.

The X-ray diffraction pattern of the test substance corresponds to the reference substance if the positions and relative intensities of the strong and medium strong bands are congruous and no additional peaks and no amorphous background appears in comparison to the reference substance.

List of significant bands: approx. 12.0°, 13.5°, 16.6°, 17.1°, 17.2°, 20.1°, 22.5°, 27.4°, 28.1°

A further X-ray powder diffraction (XRPD) has been performed with another batch of LAF237 crystal form A

XRPD-method

| | |
|---|---|
| Instrument | X1 or XDS2000; Scintag INC |
| Irradiation | CuKα (45 kV, 40 mA) |
| CuKα₁ | λ = 1.540598 Å |
| Divergence slice | 3 mm and 2 mm |
| Measuring slice | 0.3 mm and 0.2 mm |
| Chopper | 0.02 grd |
| Scan type | Continuous scan |
| Scan rate | 0.5/min (2 theta value) |
| Instrument | Stoe Powder Diffraction System |
| Irradiation | CuKα (50 kV, 30 mA) |
| CuKα₁ | λ = 1.540598 Å |
| Detector | Linear PSD |
| Scan mode | Transmission |
| Scan range | 2°-40° (2 theta value) |

Listing of most significant diffractions peaks of crystal A and calculated from single crystal structure

| | | | Calculated from single |
|---|---|---|---|
| | | | crystal structure of |
| Modification A Batch 0344012 | | | Modification A |
| 2Theta | d-spacing | Rel. | 2 Theta |
| (deg) | (Å) | intensity | (deg) |
| 10.17 | 8.69 | 4 | 10.26025 |
| 10.42 | 8.48 | 6 | 10.53656 |
| 11.83 | 7.48 | 19 | 11.94772 |
| 13.29 | 6.66 | 11 | 13.40835 |
| 16.47 | 5.38 | 30 | 16.58151 |
| 16.96 | 5.22 | 65 | 17.06789 |
| 17.14 | 5.17 | 100 | 17.26680 |
| 17.55 | 5.05 | 3 | 17.67012 |
| 18.17 | 4.88 | 14 | 18.31593 |
| 19.04 | 4.66 | 4 | 19.17206 |
| 19.58 | 4.53 | 3 | 19.68596 |
| 19.97 | 4.44 | 13 | 20.07397 |
| 20.51 | 4.33 | 4 | 20.60393 |
| 21.76 | 4.08 | 6 | 21.16355 |
| 22.28 | 3.99 | 12 | 22.36650 |
| 22.64 | 3.92 | 2 | 22.75646 |
| 23.91 | 3.72 | 3 | 24.02785 |
| 24.32 | 3.66 | 3 | 24.42784 |
| 24.69 | 3.60 | 4 | 24.76120 |
| 25.73 | 3.46 | 4 | 25.73153 |
| 26.15 | 3.41 | 3 | 26.26013 |
| 26.46 | 3.37 | 4 | 26.47020 |
| 27.16 | 3.28 | 10 | 27.18055 |
| 27.93 | 3.19 | 9 | 27.86469 |
| 29.12 | 3.06 | 4 | 29.21585 |
| 31.13 | 2.87 | 4 | 31.12078 |

### ii) IR Spectrum in Liquid Paraffin (Nujol)

Reagent used: Liquid paraffin (Nujol) for spectroscopy, e.g. Uvasol Merck No. 107161. KBr or NaCl plates.
Equipment: IR spectrophotometer e.g. Perkin-Elmer 1725-X or Bruker IFS-55.

Procedure: Mull the test substance (reference substance as needed) with liquid paraffin and record the spectrum in a minimum range of 4000-600 cm⁻¹.

If the main absorption bands are too intensive or if the baseline is too inclined due to excessive light-scattering, the preparation has to be repeated with a lower concentration.

Evaluation/assessment: Compare the positions and the relative intensities of the bands in the spectrum of the test substance with those in the spectrum of the reference substance. The spectrum of the test substance corresponds to that of the reference substance if the positions and the relative intensities of bands are concordant.

### List of Significant Bands:

| Wavenumber (cm−1) | Assignments |
|---|---|
| 3293 | ν O—H and ν N—H |
| 2925-2853 | ν CH aliphatic of nujol |
| 2238 | ν CN (nitrile) |
| 1658 | ν C═O tertiary amide |
| 1455/1354 | δ CH aliphatic of nujol |
| 1254 | ν C—N |
| 1121 | ν C—O (H) |
| 1054-1035 | ν C—O (H) cycloalkane 3-hydroxyladamantan |

ν = stretching vibration
δ = deformation vibration

The analysis of another batch of LAF237 crystal form A resulted in the following list of significant bands

| Wavenumber (cm⁻¹) | Assignments |
|---|---|
| ~3380 (broad)/3294 | ν O—H and ν N—H |
| 2993/2915/2849 | ν CH aliphatic |
| 2238 | ν CN (nitrile) |
| 1657 | ν C═O tertiary amide |
| 1405/1354 | δ CH aliphatic |
| 1254 | ν C—N |
| 1120/1102 | ν C—O (H) |
| 1054/1034 | ν C—O (H) cycloalkane 3-hydroxyladamantan |

ν = stretching vibration
δ = deformation vibration

iii) Crystallographic Analysis

The single crystal structure of LAF237-NXA, Modification A, has been successfully elucidated by the standard Crystallographic analysis.

A Nonius CAD4 automatic diffractometer was used for data collection with CuKα radiation and a graphite monochromator. The structure was solved by direct methods (SHELXS). The parameters were refined by full-matrix least square calculation (SHELXL) with anisotropic displacement parameters for all non-H atoms. A subsequent difference Fourier map showed the positions of all 25 hydrogen atoms. The parameters of the H atom were taken from the difference map and kept fixed. All the other hydrogen atom parameters were idealized and not refined. The absolute configuration was given by the synthesis.

### Crystal Data and Refinement Details of LAF237 Modification A

| sample ref. | LAF237 |
|---|---|
| chemical formula | C17H25N3O2 |
| fw | 303.40 |
| crystal size, mm | 0.59 × 0.45 × 0.32 |
| crystal system | orthorhombic |
| space group | P2₁2₁2₁ |
| a, Å | 10.263(1) |
| b, Å | 10.684(1) |
| c, Å | 14.564(1) |
| V, Å3 | 1596.9(2) |
| Z | 4 |
| D(calc), g/cm3 | 1.262 |
| radiation, Å | 1.54178 (CuKα) |
| intensity decay, % | ±1 |
| ∝, mm−1 | 0.669 |
| ∪ range from data collection, ° | 3-74.0 |
| no. of variables | 199 |
| no. of refections measured | 3547 |
| no. of reflections in least squares | 3222 |
| R | 0.065 |
| largest diff. peak/hole | 0.381/−0.245 |

Three different types of C—N bonds can be distinguished in the molecule: C—N single bonds with lengths between 1.462 Å and 1.475 Å, an amide C—N bond of 1.352 Å and a C—N triple bond of 1.129 Å. The nitrogen atom N4 is sp3 hybridised. Its lone pair is involved in an intermolecular hydrogen bond as a proton acceptor.

The six-membered rings of the adamantane moiety adopt nearly perfect chair conformations. The pyrrolidine ring has a slightly distorted envelope form with C8. 585 Å out of the plane through the other four ring atoms.

TABLE
Crystallographic data of LAF237 base.

| | |
|---|---|
| radiation, Å | 1.5406 |
| Crystal system | Orthorhombic |
| Space group | P2₁2₁2₁ |
| a, Å | 10.263(1) |
| b, Å | 10.684(1) |
| c, Å | 14.564(1) |
| V_{cell}, Å³ | 1596.9(2) |
| Z | 4 |
| D_{calc}, g cm⁻³ | 1.262 |
| N—H · · · Oᵢₙₜᵣₐ^{a} | 2.691 Å, 109° |
| O—H · · · N^{a} | 3.134 Å, 175° |
| C—H · · · O^{a} | 3.361 Å, 137° |
| C—H · · · N^{a} | 3.525 Å, 167° |

^{a}for each X—H · · · Y hydrogen bond, the X · · · Y distance and the X—H · · · Y angle are reported.

The crystal lattice of LAF237 base is characterised by an orthorhombic unit cell with two almost equal a and b axes of ca. 10 Å, the space group being P2₁2₁2₁. Bond lengths and angles are within the standard values. The amino NH group is engaged in a short intramolecular hydrogen bond with the adjacent carbonyl oxygen, see above Table for the N . . . O and N—H . . . O values. Since this nitrogen atom is sp³ hybridised, its lone pair is hydrogen bond acceptor in a O—H . . . N intermolecular contact (O . . . N=3.134 Å, O—H . . . N=175°), which runs along the [001] crystallographic direction. There are two other weak interactions in the solid state, a C—H . . . N contact along the c axis, and a C—H . . . O hydrogen bond which binds molecules in the a direction. Such isotropic distribution of intermolecular contacts indicates that LAF237 base is very stable in the solid state.

This compound forms a three dimensional network of hydrogen bonding in the crystal lattice, which indicates that this compound is very stable as crystalline phase. Comparison of simulated and experimental powder patterns could show that the current batch is a pure phase. Morphology prediction and experimental characterisation by SEM gives some discrepancy, which has been rationalised in terms of solvent effect. If LAF237 base is grown from 2-propanol, the final morphology is prismatic rather than hexagonal (as in 2-butanone), due to a good stabilisation of the (002) face with respect to the (011) one.

### 3. Water Sorption/Desorption Isotherm

Sorption/desorption isotherms were measured using Surface Measurement Systems dynamic vapor sorption device (DVS-1). Measurements were carried out at 25° C. This technique measures the sample weight as a function of relative humidity (RH). Crystalline vildagliptin Form A is only very slightly hygroscopic since it gains only 0.9% moisture at 85% RH while the amorphous sample is hygroscopic and gains 4.2% moisture at 85% RH. Forms that are hygroscopic need to be protected from the air so that they do not absorb moisture. Moisture can cause problems with formulation, stability and analysis. Thus our new crystalline vildagliptin Form A shows an additional advantage over the known vildagliptin amorphous form. As vildagliptin is highly water soluble, the use of crystalline vildagliptin “Form A” provides improved stability of the active ingredient in the galenic formulation.
AUC area under the concentration time curve
AUC₀₋ₜ The area under the plasma concentration-time curve from time zero to the last quantifiable data point t [ng*hr/mL]
AUC_{0-inf} or The area under the plasma concentration-time curve from time zero to infinity [ng*hr/mL]
AUC_{(0-∞)}
BAPK Bioanalytics and Pharmacokinrtics section
Cₘₐₓ maximum plasma concentration
CRF case report/record form
CRO Clinical Research Organization
CV Coefficient of variation
ECG Electrocardiogram
DPP-4 dipeptidyl peptidase 4; dipeptidyl peptidase IV
FMI Final Market Image
GLP-1 glucagon-like peptide 1
ICH International Council on Harmonization
IRB Institutional Review Board
LAF237 Vildagliptin
LC-MS/MS Liquid chromatography-mass spectrometry/mass spectrometry
LOQ limit of quantitation
o.d. once a day
PD Pharmacodynamics
PK Pharmacokinetics
p.o per os/by mouth/orally
QC Quality Control
SOP Standard Operating Procedures
SD standard deviation
tₘₐₓ time to reach C.
t_{1/2} elimination half-life
Vd/f volume of distribution, corrected for the absolute bioavailability

### EXAMPLE 8

### The DPP-4 Inhibition Activity has been Obtained Out of Clinical Studies as Described Below

Study title: A randomized, open-label, placebo-controlled, seven-period, crossover study to evaluate dose-response relationship following single oral doses of a 10, 25, 50, 100, 200, and 400 mg of a Vildagliptin formulation in type 2 diabetics that are challenged with 75-gm oral glucose tolerance test. Vildagliptin is administered with the herein described dosage forms i.e. formulations, tablets and capsules.

### Objectives:

To evaluate the dose-dependent effects of Vildagliptin on DPP-IV inhibition in type 2 diabetic subjects during 75-gm oral glucose tolerance test.

Design: This was a randomized, open-label, placebo-controlled, seven period, crossover study. Fourteen type 2 diabetic subjects completed the study. There was a 29-day screening period including a 21-day washout from prior hypoglycemic agents. Subjects previously on metformin therapy were required to undergo a 28-day washout. Subjects had an average fasting plasma glucose of 7.0-10 mmol/L (126-180 mg/dl), representing the mean of 3 measures taken on 3 separate days during the last 2 weeks before dosing. HbA1c at screening was 7.5-10%.

Eligible subjects were randomized to one of fourteen sequences. There was a 36-hr baseline period prior to first dose, a minimum 3 week domiciled stay toward the completion of 7 treatment periods, a study completion evaluation following the last pharmacodynamic assessment. Subjects consumed standardized BDA meals and had baseline evaluations on Day-1.

The inter-dose interval was 72 hr. On dosing days, subjects were administered the assigned dose following an overnight fast. Subjects consumed a 75-gm oral glucose load 30 min following the dose. Pharmacokinetic and pharmacodynamic sampling occurred at specified times.

On dosing days, subjects skipped the breakfast meal. Standard lunch and dinner meals were consumed at 5.5 hr and 10 hr postdose, respectively. During the remaining domiciled days, subjects maintained a standard BDA diet. End of study evaluations occurred following the completion of the last pharmacodynamic assessments for treatment period 7.

### Number of Subjects:

Fourteen subjects were to complete the study. In total, sixteen subjects were dosed in this study. Of these, 2 subjects discontinued and 14 subjects completed the study.

### Criteria for Inclusion:

Male and non-fertile females (i.e., post-menopausal, post-hysterectomy, or sterilized by tubal ligation) with type 2 diabetes with at least 3 months of disease duration, 30 and 70 years of age, and willing to undergo a 3-week hypoglycemic washout. The average fasting plasma glucose from 3 assessments completed in the last two weeks of washout should be between 7.0 to 10 mmol/L (126-180 mg/dL), HbA_{1c} at screening from 7.5-10%, C-peptide≧0.3 nmol/L, and body mass index 40 kg/m².

Investigational drug: Vildagliptin

Duration of treatment: Subjects were randomized to receive a single dose each of 10 mg, 25 mg, 50 mg, 100 mg, 200 mg, 400 mg Vildagliptin, and placebo per treatment period. The inter-dose interval between consecutive treatments was 72 hr.

### Criteria for Evaluation:

Safety and tolerability: Safety and tolerability assessments consisted of vital signs, ECGs, biochemistry, hematology, and urinalysis as specified below.
Hematology; Blood chemistry; Urinalysis: screening, baseline, predose to Periods 3 and 5, and study completion evaluation
Hemoccult: Screening, baseline, Periods 3, 5, and study completion.
Adverse events; Concomitant medications/Significant non-drug therapies: from time of first administration of study drug until end of study

### Pharmacokinetics:

### Time of OGTT is Considered 0 hr

Blood collection for LAF237 determination [1 mL blood per sample, heparin tubes (plasma)]: −0.5 hr (prior to Vildagliptin dose), 0.5, 1.5, 5, and 8 hr post-OGTT
Analytes, media and methods: Vildagliptin in plasma by LC-MS/MS; LOQ of approximately 2 ng/mL
PK parameters for LAF237: AUC, AUC₀₋ₜ, t_{1/2}, Cₘₐₓ, tₘₐₓ, CL/F

### Pharmacodynamics:

### AM Dose at ˜0800 hr

AM OGTT at ˜0830 hr following dose
Note: All PD times listed below are w.r.t. OGTT

### Plasma DPP-IV Peptidase Activity (1 mL Blood Sample)

On each Treatment Day:

1 hr and 0.75 hr prior to OGTT

Following OGTT: −0.25, 0 (prior to OGTT), 0.25, 0.5, 1, 1.5, 2, 4, 6, 8, 10, 12, 16, and 24 hr.

### Statistical Methods:

Statistical comparisons of the pharmacodynamic parameters AUE and Eₘₐₓ for glucose, insulin, glucagon, and GIP are made based on analysis of variance. For both parameters, the log-transformed data are analyzed using a linear mixed effect model including treatment, period and sequence as fixed factors and patient within sequence as a random factor. A point estimate and a 90% confidence interval for the ratio of treatment means on the original scale are provided for each comparison. The comparison between treatment group and the placebo group is a primary analysis. Additional analyses are also conducted to compare among active treatment groups.

### Data Analysis

The DPP-4 activity is measured before and after vildagliptin administration at various time point until 24 hr. FIG. 7 illustrate the percentage DPP-4 inhibition. The percentage of DPP-4 inhibition is calculated from the measured DPP-4 activity by the following equation:

$$DPP \text{-} {4}_{inhibition} ( \% ) = [ 1 - \frac{DPP \text{-} 4 activity ( t )}{DPP \text{-} 4 activity ( 0 )} ] \times 100 \qquad ( 1 )$$

Where DPP-4-activity(t) is the measured DPP-4 activity at time t, and DPP-4-activity(0) is the baseline DPP-4 activity measured before the administration of vildagliptin.

The mean residence time (MRT) of DPP-4 inhibition is estimated from the DPP-4 percentage inhibition vs. time profile after each dosing regimen based on the non-compartmental analysis using WinNonlin (ver 4.1, Pharsight, Calif.). The mean residence time of DPP-4 inhibition was estimated with the following equation:

$$MRT = \frac{{\int }_{0}^{24} DPP \text{-} 4 inhibition ( t ) \times d t}{{\int }_{0}^{24} DPP \text{-} 4 inhibition ( t ) d t} \qquad ( 2 )$$

The average DPP-4 inhibition over 24-hr interval is estimated by dividing the area under the DPP-4 percentage vs. time profiles by the time interval. The following equation was used to calculate the average DPP-4 inhibition over 24-hr:

$$AverageDPP \text{-} 4 {inhibition}_{0 - 24} = \frac{{\int }_{0}^{24} DPP \text{-} 4 inhibition ( t ) d t}{24} \qquad ( 3 )$$

### EXAMPLE 9

An open-label, single-dose, four-period, four-treatment, randomized crossover study with a 2-day washout between each period to compare the plasma concentrations of 25, 50, 100 and 200 mg of vildagliptin (with the herein described formulations, tablets and capsules) in healthy volunteers is carried out. A total of 20 healthy subjects enrolled and 20 completed all study procedures and treatments. Subjects are screened during a 21-Day period and, if eligible, proceeded to a baseline visit prior to each treatment (four baseline evaluations in total). There is an end of study evaluation prior to discharge from the study site. Subjects are randomized into 4 dosing sequence groups with 5 subjects per sequence. The subjects are admitted to the study center at least 12 hours prior to the initial dosing in each period for baseline evaluations, and are confined to the clinic for at least 24 h post-dose in each period. Following an inter-dose interval of at least 2 days, each subject returned to the study site to receive the alternate treatment as per their randomization schedule. All subjects receive each of the 25, 50, 100 and 200 mg treatments once during the study according to a randomization schedule.

Plasma samples for determination of vildagliptin are obtained over 24 hour period after the dose in each treatment segment. For all treatment periods, subjects fasted for a minimum of 10 hours pre-dose to 4 hours post-dose. Subjects are considered to have completed the study when all safety and pharmacokinetic evaluations had been completed.

Blood samples are collected to determine the pharmacokinetics following a single oral dose of 25, 50, 100 or 200 mg of vildagliptin. Plasma concentrations of vildagliptin are used to determine the pharmacokinetic parameters using non-compartmental methods, and the data are summarized in FIGS. 3 and 4, and Table 1.

TABLE 1
Arithmetic mean of pharmacokinetic parameters of vildagliptin following
a single oral administration of 25, 50, 100 and 200 mg FMI tablets

| | | | | AUC₀₋ₜ | | AUC_{0-∞} | | |
|---|---|---|---|---|---|---|---|---|
| | tₘₐₓ (h) | Cₘₐₓ (ng/mL) | | (h · ng/ml) | | (h · ng/mL) | | t_{1/2} (h) |
| Dose | median | mean ± | | mean ± | | mean ± | | mean ± |
| (mg) | (min, max) | SD (CV %) | | SD (CV %) | | SD (CV %) | | SD (CV %) |
| 25 | 1.5 (1.0, 6.0) | 117 ± 41 | (35) | 453 ± 91 | (20) | 461 ± 91 | (20) | 1.7 ± 0.35 (21) |
| 50 | 1.5 (0.5, 6.0) | 245 ± 87 | (36) | 1020 ± 193 | (19%) | 1030 ± 191 | (19) | 2.3 ± 1.42 (62) |
| 100 | 1.75 (0.75, 6.0) | 505 ± 120 | (24) | 2330 ± 278 | (12%) | 2350 ± 279 | (54) | 2.5 ± 1.34 (54) |
| 200 | 1.25 (0.75, 4.0) | 1100 ± 280 | (26) | 5060 ± 722 | (14) | 5080 ± 721 | (14) | 3.1 ± 1.06 (34) |

### EXAMPLE 10

An open-label, 3-period study in patients with type 2 diabetes is carried out to evaluate the pharmacokinetic drug-drug interaction between vildagliptin 100 mg qd and metformin 1000 mg qd when given alone or in combination for 5 days. A total of 17 patients are enrolled and all completed all study procedures and treatments. The subjects are admitted to the study center for at least 12 hours prior to the initial dosing for baseline evaluation, and confined to the clinic for entire study. Subjects are given an end-of-study evaluation on the last day of period 2 (Day 20). For all treatment periods, subjects fasted for a minimum of 10 hours pre-dose to 4 hours post-dose. Subjects are considered to have completed the study when all safety and pharmacokinetic evaluations have been completed.

Pharmacokinetic blood sampling for vildagliptin and metformin are collected over 24-hr for pharmacokinetic evaluation. The pharmacokinetic profiles are illustrated in FIG. 5. Pharmacokinetic parameters are determined using non-compartmental methods, and the data are summarized in Table 2.

TABLE 2
Pharmacokinetic parameters in patients with type 2 diabetes at steady state
following multiple dose administration of vildagliptin 100 mg qd FMI tablets

| | | | AUC₀₋₂₄ | CL/F | |
|---|---|---|---|---|---|
| | Tₘₐₓ (h) | Cₘₐₓ (ng/mL) | (h · ng/mL) | (L/h) | t_{1/2} (h) |
| | median | mean ± SD | mean ± SD | mean ± SD | mean ± SD |
| Treatment | (min, max) | (CV %) | (CV %) | (CV %) | (CV %) |
| LAF237 | 1.00 | 467 ± 134 | 1960 ± 413 | 53.3 ± 11.3 | 1.68 ± 0.259 |
| Alone | (0.50, 4.00) | (29) | (21) | (21) | (15) |
| (N = 17) | | | | | |
| LAF237 + | 2.50 | 381 ± 103 | 1840 ± 360 | 56.6 ± 12.3 | 1.86 ± 0.689 |
| Metformin | (0.50, 4.00) | (27) | (20) | (22) | (37) |
| (N = 17) | | | | | |

### EXAMPLE 11

The study is an open-label, three-period, multiple dose design to evaluate the pharmacokinetic drug-drug interaction between vildagliptin 100 mg qd and pioglitazone 45 mg qd when given alone or in combination to patients with type 2 diabetes after multiple dosing for 28 or 7 days. After screening, a total of 15 patients are enrolled and all completed the study. The subjects are admitted to the study center at least 12 hours prior to the initial dosing for baseline evaluation. If subjects met all eligibility criteria at baseline, they are randomized into the study. All study medications are taken 30 minutes before breakfast. The study is completed with the end of study evaluations on the last day of treatment period.

Pharmacokinetic blood sampling for vildagliptin is collected over 24-hr on day 7 and day 28, respectively, when given with alone or in combination with pioglitazone. Pharmacokinetic profiles of vildagliptin when given a lone or in combination with pioglitazone are illustrated in FIG. 6. Pharmacokinetic parameters are determined using non-compartmental methods, data shown in Table 3.

TABLE 1
Pharmacokinetic parameters of vildagliptin at steady-state
when given alone or in combination with pioglitazone

| | | | AUC₀₋₂₄ | AUC_{0-inf} | | |
|---|---|---|---|---|---|---|
| | Tₘₐₓ (hr) | Cₘₐₓ (ng/mL) | (ng/mL × hr) | (ng/mL × hr) | t_{1/2} (hr) | CL/F (L/hr) |
| | Median | Mean ± SD | Mean ± SD | Mean ± SD | Mean ± SD | Mean ± SD |
| Treatment | (Min, Max) | (CV %) | (CV %) | (CV %) | (CV %) | (CV %) |
| LAF237 | 1.75 | 531 ± 115 | 2190 ± 425 | 2210 ± 425 | 3.71 ± 2.14 | 47.5 ± 10.3 |
| alone | (0.5-4.0) | (22) | (19) | (19) | (58) | (22) |
| LAF237 + | 1.00 | 505 ± 117 | 2080 ± 448 | 2090 ± 446 | 3.82 ± 1.64 | 50.6 ± 12.7 |
| Pioglitazone | (0.5-4.0) | (35) | (22) | (21) | (43) | (25) |

What is claimed is:

1. A compressed pharmaceutical tablet or a direct compressed pharmaceutical tablet, comprising vildagliptin crystal “Form A” or pharmaceutical salts thereof and wherein at least 40% of the particle size distribution in the table is between 10 to 250 μm.

2. The compressed pharmaceutical tablet or a direct compressed pharmaceutical tablet of claim 1 contains particles comprising DPP-IV inhibitor, in free form or in acid addition salt form, and wherein tablet thickness to tablet weight ratios is of 0.002 to 0.06 mm/mg.

3. The compressed pharmaceutical tablet or a direct compressed pharmaceutical tablet of claim 1 containing articles comprising DPP-IV inhibitor, in free form or in acid addition salt form, and wherein:
i) at least 40% of the particle size distribution in the tablet is between 10 to 250 μm, and
ii) tablet thickness to tablet weight ratios is of 0.002 to 0.06 mm/mg or of 0.01 to 0.03 mm/mg

4. The compressed pharmaceutical tablet or a direct compressed pharmaceutical tablet of claim 1 containing particles comprising DPP-IV inhibitor, in free form or in acid addition salt form, and wherein:
i) at least 40% of the particle size distribution in the tablet is between 10 to 250 μm,
ii) the water content of the tablet is less than 10% after 1 week at 25° C., and 60% relative humidity (RH), and
iii) tablet thickness to tablet weight ratios is of 0.002 to 0.06 mm/mg.

5. A compressed pharmaceutical tablet or a direct compressed pharmaceutical tablet according to claim 1, wherein the particle size distribution in the tablet is between 50 to 150 μm.

6. A compressed pharmaceutical tablet or a direct compressed pharmaceutical tablet according to claim 1, wherein the water content of the tablet is less than 5% after 1 week at 25° C. and 60% relative humidity (RH).

7. A compressed pharmaceutical tablet or a direct compressed pharmaceutical tablet according to claim 1, wherein the ratio of the tablet thickness to tablet weight ratios is of 0.01 to 0.03 mm/mg

8. A compressed pharmaceutical tablet or a direct compressed pharmaceutical tablet according to claim 1, wherein at least 60% of the particle size distribution in the tablet is between 10 to 250 μm.

9. A compressed pharmaceutical tablet or a direct compressed pharmaceutical tablet according to claim 1, wherein at least 25% or at least 35% of the particle size distribution in the tablet is between 50 to 150 μm.

10. A compressed pharmaceutical tablet or a direct compressed pharmaceutical tablet according to claim 1, wherein the tablet comprises a further therapeutic agent.

11. A compressed pharmaceutical tablet or a direct compressed pharmaceutical tablet according to claim 1, wherein
i) between 0 and 10 minutes 85 to 99.5% of the active ingredient is released, and
ii) between 10 and 15 minutes 90 to 99.5% of the active ingredient is released:
wherein release is measured using liquid chromatography-mass spectrometry/mass (LC/MS-MS).

12. The compressed pharmaceutical tablet or a direct compressed pharmaceutical tablet of claim 1 wherein:
i) at least 40%, of the particle size distribution is between 10 to 250 μm, and/or
ii) at least 60% of the particle size distribution is between 10 to 250 μm, and/or
iii) at least 25% or at least 35% of the particle size distribution is between 50 to 150 μm.

13. A compressed pharmaceutical tablet or a direct compressed pharmaceutical tablet according to claim 1 comprising between 20 and 120 mg of vildagliptin or a pharmaceutically acceptable acid addition salt thereof.

14. The compressed pharmaceutical tablet or a direct compressed pharmaceutical tablet of claim 1 which further comprises:
(c) 0-20% by weight on a dry weight basis of a pharmaceutically acceptable disintegrant;
(d) 0.1-10% by weight on a dry weight basis of a pharmaceutically acceptable lubricant.

15. The composition of claim 14, which further comprises:
(c) 1-6% by weight on a dry weight basis of a pharmaceutically acceptable disintegrant; and
(d) 0.25-6% by weight on a dry weight basis of a pharmaceutically acceptable lubricant.

16. The composition of claim 14, which further comprises:
(c) 1-4% by weight on a dry weight basis of a pharmaceutically acceptable sodium starch glycolate; and
(d) 0.5-4% by weight on a dry weight basis of magnesium stearate.

17. A pharmaceutical composition comprising the compressed pharmaceutical tablet or a direct compressed pharmaceutical tablet of claim 1.

18. The compressed pharmaceutical tablet or a direct compressed pharmaceutical tablet of claim 1, wherein
i) between 0 and 10 minutes 85 to 99.5% of the active ingredient is released, and
ii) between 10 and 15 minutes 90 to 99.5% of the active ingredient is released, or,
i) between 0 and 10 minutes 88 to 99.5% of the active ingredient is released, and
ii) between 10 and 15 minutes 95 to 99.5% of the active ingredient is released, or
i) between 0 and 10 minutes 89 to 94% of the active ingredient is released, and
ii) between 10 and 15 minutes 96 to 99% of the active ingredient is released, in a 0.01N HCl solution.
Wherein release is measured using liquid chromatography-mass spectrometry/mass (LC/MS-MS).

19. The compressed pharmaceutical tablet or a direct compressed pharmaceutical tablet of claim 1 wherein the composition comprises a further therapeutic agent.

20. The compressed pharmaceutical tablet or a direct compressed pharmaceutical tablet of claim 1 in immediate release dosage form, wherein the average DPP-IV inhibition, 10.5 hours after a once daily administration of 50 mg of vildagliptin or a salt thereof in patients with type 2 diabetes, is at least 79.

21. The compressed pharmaceutical tablet or a direct compressed pharmaceutical tablet of claim 1 in immediate release dosage form, wherein the average DPP-IV inhibition, between 0.25 and 10.5 hours after a once daily administration of 50 mg of vildagliptin or a salt thereof, in patients with type 2 diabetes, is between 84% and 98%.

22. The compressed pharmaceutical tablet or a direct compressed pharmaceutical tablet of claim 1 in immediate release dosage form, wherein the average DPP-IV inhibition over 24 hours after a once daily administration of 50 mg of vildagliptin or a salt thereof, in patients with type 2 diabetes, is of 64.2%+1-12.7%.

23. The compressed pharmaceutical tablet or a direct compressed pharmaceutical tablet of claim 1 in immediate release dosage form, wherein the average DPP-IV inhibition, 10.5 hours after a once daily administration of 100 mg of vildagliptin or a salt thereof, in patients with type 2 diabetes, is at least 83%.

24. The compressed pharmaceutical tablet or a direct compressed pharmaceutical tablet of claim 1 in immediate release dosage form, wherein the average DPP-IV inhibition, between 0.25 and 10.5 hours after a once daily administration of 100 mg of vildagliptin or a salt thereof, in patients with type 2 diabetes, is between 84% and 98.8%.

25. The compressed pharmaceutical tablet or a direct compressed pharmaceutical tablet of claim 1 in immediate release dosage form, wherein the average DPP-IV inhibition over 24 hours after a once daily administration of 100 mg of vildagliptin or a salt thereof, in patients with type 2 diabetes, is of 76.3%+1-13.7%.

26. The compressed pharmaceutical tablet or a direct compressed pharmaceutical tablet of claim 1 comprising about 50 mg vildagliptin, or a respective amount of a pharmaceutically acceptable salt thereof, and a carrier medium, wherein said tablet provides:
an arithmetic mean maximum plasma concentration of vildagliptin ranging from about 77.3 ng/mL+/−20.8 ng/mL to about 195 ng/mL+/−89.1 ng/mL between about 0.5 and about 6 hours following oral administration of a single 50 mg dose of vildagliptin, and/or
an arithmetic mean AUC_{(0-∞)} of vildagliptin ranging from about 839 to about 1221 ng·h/mL i.e. 1030 ng·h/mL+/−191 ng·h/mL following oral administration of a single dose of 50 mg of vildagliptin, and/or
an arithmetic mean tₘₐₓ of vildagliptin of 2.1 hr+/−1.3 hr following oral administration of a single dose of 50 mg of vildagliptin.

27. The compressed pharmaceutical tablet or a direct compressed pharmaceutical tablet of claim 1 comprising about 100 mg vildagliptin, or a respective amount of a pharmaceutically acceptable salt thereof, and a carrier medium, wherein said tablet provides:
an arithmetic mean maximum plasma concentration of vildagliptin ranging from about 186 ng/mL+/−64.9 ng/mL to about 428 ng/mL+/−165 ng/mL between about 0.5 and about 6 hours following oral administration of a single 50 mg dose of vildagliptin, and/or
an arithmetic mean AUC_{(0-∞)} of vildagliptin ranging from about 2071 to about 2629 ng·h/mL i.e. 2350 ng·h/mL+/−279 ng·h/mL following oral administration of a single dose of 100 mg of vildagliptin, and/or
an arithmetic mean tₘₐₓ of vildagliptin of 2.0 hr+/−1.4 hr following oral administration of a single dose of 100 mg of vildagliptin.

28. The compressed pharmaceutical tablet or a direct compressed pharmaceutical tablet of claim 27, wherein the administration of the tablet is performed in a healthy human subject.

29. The compressed pharmaceutical tablet or a direct compressed pharmaceutical tablet of claim 1 comprising about 100 mg vildagliptin, or a respective amount of a pharmaceutically acceptable salt thereof, and a carrier medium, wherein said dosage form provides:
an arithmetic mean maximum plasma concentration of vildagliptin ranging from about 1×8 ng/mL+/−132 ng/mL to about 327 ng/mL+/−87.6 ng/mL between about 0.5 and about 6 hours following oral administration of a single 100 mg dose of vildagliptin, concomitantly with 1000 mg of metformin, and/or
an arithmetic mean AUC_{(0-24 h)} of vildagliptin of 1840 ng·h/mL+/−360 ng·h/mL following oral administration of a single dose of 100 mg of vildagliptin, concomitantly with 1000 mg of metformin, and/or
an arithmetic mean tₘₐₓ of vildagliptin of 2.5 hr+/−1.3 hr following oral administration of a single dose of 100 mg of vildagliptin, concomitantly with 1000 mg of metformin.

30. The compressed pharmaceutical tablet or a direct compressed pharmaceutical tablet of claim 1 comprising about 100 mg vildagliptin, or a respective amount of a pharmaceutically acceptable salt thereof, and a carrier medium, wherein said tablet provides:
an arithmetic mean maximum plasma concentration of vildagliptin ranging from about 123 ng/mL+/−51.5 ng/mL to about 455 ng/mL+/−217 ng/mL between about 0.5 and about 6 hours following oral administration of a single 100 mg dose of vildagliptin, concomitantly with 45 mg of pioglitazone, and/or,
an arithmetic mean AUC_{(0-∞)} of vildagliptin of 2090 ng·h/mL+/−446 ng·h/mL following oral administration of a single dose of 100 mg of vildagliptin, concomitantly with 45 mg of pioglitazone, and/or
an arithmetic mean tₘₐₓ of vildagliptin of 1 hr+/−1.3 hr following oral administration of a single dose of 100 mg of vildagliptin, concomitantly with 45 mg of pioglitazone.

31. The compressed pharmaceutical tablet or a direct compressed pharmaceutical tablet of claim 1, wherein the oral dosage is performed in a human subject with type 2 diabetes.

32. The compressed pharmaceutical tablet or a direct compressed pharmaceutical tablet of claim 1 comprising vildagliptin crystal “Form A” or pharmaceutical salts thereof having a particle size distribution of between 10 to 250 μm, the vildagliptin crystal “Form A” being identified by:
(i) an X-ray diffraction pattern having significant bands of approximately 12.0°, 13.5°, 16.6°, 17.1°, 17.2°, and 20.1°;
(ii) an IR spectrum having absorption bands expressed in reciprocal wve numbers (cm⁻¹) of 2238 and 1254; and
(iii) comprising at least three different types of C—N bonds, the bonds being a C—N single bond of between about 1.462 A and 1.475 A, an amide C—N bond of about 1.352 A, and a C—N triple bond of about 1.129 A.
